SEQUENCE LISTING (303 sequences)

SEQ ID NO: 1 - protein, 13 aa, Homo sapiens
  PEPTIDE (1)..(13)
  PEPTIDE (1)..(13): BUC11 fragment
PSKRLFFKKKRLC

SEQ ID NO: 2 - DNA, 20 bp, Artificial Sequence
  Primer for BUC6
tggaaaatgtgtccaccaag

SEQ ID NO: 3 - DNA, 20 bp, Artificial Sequence
  Primer for BUC6
cgctgctgtaagcattcact

SEQ ID NO: 4 - DNA, 20 bp, Artificial Sequence
  Primer for BUC 6
ttgcataaagctgcacagga

SEQ ID NO: 5 - DNA, 20 bp, Artificial Sequence
  Primer for BUC6
cttcaacacaacccccatgt

SEQ ID NO: 6 - DNA, 21 bp, Artificial Sequence
  Primer for BUC 6
ccttcgagttcctttttctgg

SEQ ID NO: 7 - DNA, 20 bp, Artificial Sequence
  Primer for BUC6
ggcaacacaaactcagagca

SEQ ID NO: 8 - DNA, 20 bp, Artificial Sequence
  Primer for BUC9
ccagattttcaccgctatgc

SEQ ID NO: 9 - DNA, 20 bp, Artificial Sequence
  Primer for BUC9
aggcaagctctcatcaggac

SEQ ID NO: 10 - DNA, 20 bp, Artificial Sequence
  Primer for BUC10
caccgacgtttaaaggagga

SEQ ID NO: 11 - DNA, 20 bp, Artificial Sequence
  Primer for BUC10
gtcctctgcaccttgggata

SEQ ID NO: 12 - DNA, 20 bp, Artificial Sequence
  Primer for BUC11
tctttcccacaatccctgac

SEQ ID NO: 13 - DNA, 20 bp, Artificial Sequence
  Primer for BUC11
cagcttgccccatgtatttt

SEQ ID NO: 14 - DNA, 20 bp, Artificial Sequence
  Primer  for BUC11
tgccattccactgttttctg

SEQ ID NO: 15 - DNA, 20 bp, Artificial Sequence
  Primer for BUC11
cagcttgccccatgtatttt

SEQ ID NO: 16 - DNA, 30 bp, Artificial Sequence
  Primer for BUC11
tctagtcgacccacaatccctgacacagaa

SEQ ID NO: 17 - DNA, 30 bp, Artificial Sequence
  Primer for BUC11
tctatctagagccagaaaacagtggaatgg

SEQ ID NO: 18 - DNA, 20 bp, Artificial Sequence
  Primer for BUC6/9
gaggccttgctaatttccta

SEQ ID NO: 19 - DNA, 20 bp, Artificial Sequence
  Primer for BUC6/9
ctgttgcagtgagctcaagt

SEQ ID NO: 20 - DNA, 20 bp, Artificial Sequence
  Primer for BUC9/10
gtcctgatgagagcttgcct

SEQ ID NO: 21 - DNA, 20 bp, Artificial Sequence
  Primer for BUC9/10
caaactggccttgatctgga

SEQ ID NO: 22 - DNA, 20 bp, Artificial Sequence
  Primer for BUC6Q
ctcgaagccatcaatgacaa

SEQ ID NO: 23 - DNA, 20 bp, Artificial Sequence
  Primer for BUC6Q
tgagataatccgctccttgg

SEQ ID NO: 24 - DNA, 20 bp, Artificial Sequence
  Primer for BUC9Q
gccacatggggtatgttctc

SEQ ID NO: 25 - DNA, 20 bp, Artificial Sequence
  Primer for BUC9Q
aggcaagctctcatcaggac

SEQ ID NO: 26 - protein, 75 aa, Homo sapiens
MGLLPPPSKRLFFKKKRLCFDLKYFIYPQSQDTVDGIKAKHICKKDIAGGGAKSWKPSLQSWEKQWYGKAMERAF

SEQ ID NO: 27 - DNA, 20 bp, Artificial Sequence
  GADPH primer
gagtcaacggatttggtcgt

SEQ ID NO: 28 - DNA, 20 bp, Artificial Sequence
  GADPH primer
aatgaaggggtcattgatgg

SEQ ID NO: 29 - protein, 13 aa, Homo sapiens
IDRHQHTRFIADS

SEQ ID NO: 30 - protein, 21 aa, Homo sapiens
RRMFMLGHRTQVEREELWVRL

SEQ ID NO: 31 - DNA, 2420 bp, Homo sapiens
aatctcgctctgtcactcaggctggagtgcagtggcgcaatctcggctcactgcaaactctgcctcccaggttcacgccattctcctgccacagcctcccgagtagctgggactacaggcgcccgccaccaagcacagctaagttttttatttatagtagagacggggtttcactgtgttagccaggatggtctcgatctcctgacctcgtgatctgcctgcctcggcctcccaaagtgccgggattacaggcgtgagccaccgcgccgggcctgatttcagtttcttccagcccttcctattgttaacatgggggttgtgttgaagaatataaagttacaaagtcaaggaagtaggaaacatttttacaagtattatgtagccatcttggtggggctgtggtgaggtaggctgcaaatgattctcctatttctttccctgagttcagaacataggaattagattgatagacatcaacatacccgctttattgctgactcatgacaactaatgggaagacatggctcagatgtgcagccacagtgagcttctgaacatttcttctcagactaagctcttacacacagttgcagttgaaagaaagaattgcttgacatggccacaggagcaggcagcttcctgcagacatgacagtcaacgcaaactcatgtcactgtgggcagacacatgtttgcaaagagactcagagccaaacaagcacactcaatgtgctttgcccaaatttacccattaggtaaatcttccctcctcccaagaagaaagtggagagagcatgagtcctcacatggaaacttgaagtcagggaaatgaaggctcaccaattatttgtgcatgggtttaagttttccttgaaattaagttcaggtttgtctttgtgtgtaccaattaatgacaagaggttagatagaagtatgctagatggcaaagagaaatatgttttgtgtcttcaattttgctaaaaataacccagaacatggataattcatttattaattgattttggtaagccaagtcctatttggagaaaattaatagtttttctaaaaaagaattttctcaatatcacctggcttgataacatttttctccttcgagttcctttttctggagtttaacaaacttgttctttacaaatagattatattgactaccactcactgatgttatgatattagtttctattgcttactttgtatttctaattttaggattcacaatttagctggagaactattttttaacctgttgcacctaaacatgattgagctagaagacagttttaccatatgcatgcattttctctgagttatattttaaaatctatacatttctcctaaatatggaggaaatcactggcatcaaatgccagtctcagacggaagacctaaagcccatttctggcctggagctacttggctttgtgacctatggtgaggcataagtgctctgagtttgtgttgcctcttttgtaaaatgagggtttgacttaatcagtgattttcatagcttaaaatttttttgaagaacagaactttttttaaaaacagttagatgcaaccatattatataaaacagaacagatacaagtagagctaacttgctaaagaaaggatggaggctctgaagctgtgacttcattatcccttaatactgctatgtcctctgtagtaccttagatttctatgggacatcgtttaaaaactattgtttatgcgagagccttgctaatttcctaaaaattgtggatacattttttctcccatgtataattttctcaccttctatttaaaaagaaaaaaaaagtcagtgtagtatttacatattttaccctataaggagctaacataacttttgatttagtgttattcataaaattaggttagcagtttattaaccttttgtatttgctctggcaatgtttaatatctcataagctatacacacctcgaagccatcaatgacaaccttttcttgctgaatagaacagtgattgatgtcatgaagacaattttatctccttttgccttccataatttgtaccagcaggttatataatagtataacactgccaaggagcggattatctcatcttcatcctgtaattccagtgtttgtcacgtggttgttgaataaatgaataaagaatgagaaaaccagaagctctgatacataatcataatgataattatttcaatgcacaactacgggtggtgctgaactagaatctatattttctgaaactggctcctctaggatctactaatgatttaaatctaaaagatgaagttagtaaagcatcagaaaaaaaaggtgggtattcctacaagtcaggacattctacgtgactacaatataatctcacagaaatttaacattaatacattctaagatttaattct

SEQ ID NO: 32 - DNA, 771 bp, Homo sapiens
gtaaacaaattgctcctgtggagatgattggcatcacatggtgttttgagctgatacacccaacacttgagctcactgcaacagtaccagattttcaccgctatgcctcctttcactctgggagtcttccagaggtcttgcactcgggagagcatgctcaggtttccccagctctacaaaatcacccagaatgccaaagacttcaacacaagggtaaataaggttgatctcagaattgtcacctcaaaaaggccctgccttccactgttcagttctggtcatctgcctatgagatatctgaagcttgaaagagaacacttgaaaatcactgagaccgtgactcccatcccagcacacacagcaagccaagtaggttacagagatttcttcttgggtgatgagttcacgccacatggggtatgttctccagttccagtgtgttgactccttcctgcttccccccatcagcccctgaggtcaatgtgggcagcagcccccatggtccaagttcaagatccgctgtggaagacttttttaggcaatcacacagccctgcatggagtcctgatgagagcttgcctaattgttgctaggtttgtcattttaaatacagtgtttctttagctagtgagtaaaattggctatataggagatactgtgttgaccagtggtcatgccactgcctgttgatttgttgaaaatattgtttacacgtatgttcttgttactgattgtcagaaagctggttttgagactgcagcttggactaaa

SEQ ID NO: 33 - DNA, 641 bp, Homo sapiens
acgccaagctatttaggtgacactagaatactcaagctatgcatcaagcttggtaccgagctcggatccactagtaacggccgccagtgtgctggaattcgcccttccagattttcaccgctatgcctcctttcactctgggagtcttccagaggtcttgcactcgggagagcatgctcaggtttccccagctctacaaaatcacccagaatgccaaagacttcaacacaagggtaaataaggttgatctcagaattgtcacctcaaaaaggccctgccttccactgttcagttctggtcatctgcctatgagatatctgaagcttgaaagagaacacttgaaaatcactgagaccgtgactcccatcccagcacacacagcaagccaagtaggttacagagatttcttcttgggtgatgagttcacgccacatggggtatgttctccagttccagtgtgttgactccttcctgcttccccccatcagcccctgaggtcaatgtgggcagcagcccccatggtccaagttcaagatccgctgtggaagacttttttaggcaatcacacagccctgcatggagtcctgatgagagcttgcctaagggcgaattctgcagatatccatcacactggcggccgc

SEQ ID NO: 34 - DNA, 477 bp, Homo sapiens
ctagtaacggccgccagtgtgctggaattgcgcccttccagattttcaccgctatgcctcctttcactctgggagtcttccagaggtcttgcactcgggagagcatgctcaggtttccccagctctacaaaatcacccagaatgccaaagacttcaacacaagggtaaataaggttgatctcagaattgtcacctcaaaaaggccctgccttccactgttcagttctggtcatctgcctatgagatatctgaagcttgaaagagaacacttgaaaatcactgagaccgtgactcccatcccagcacacacagcaagccaacccctgaggtcaatgtgggcagcagcccccatggtccaagttcaagatccgctgtggaagacttttttaggcaatcacacagccctgcatggagtcctgatgagagcttgcctaagggcgaattctgcagatatccatcacactggcggccgctcga

SEQ ID NO: 35 - DNA, 1921 bp, Homo sapiens
ttcagtcatctggctgtctggggaagcatgctgaccagtctggtgttctttggcatctactcagccatctggtccaccattctcattgccccaaatatgagaggacaggtaagtatgccagattggaagtttgccttctgtgtcttgtgataacaatgtttctccatgtgtaggcaagcaggctctctgtgttttacaactgtattgaggtttgatagacgctggtgctacctctttctgtcaggatctatcaggaggtaaaccaaattaaagcacctggaagatgtcatccaatccacagaaatggcatttcaaatttaaagtattataaaaagtcaacctaaatccccttacttacctggcatgtccacattttcataataaattatgtgttaattgcacatagtactgatatgtgcagtttatggccctttggaaactggagctattttgagttgctcataatccagtgattatactggttacagttttgatgttgtactctgattagtagttatcctctgctgagtcatctgtggaagttaaatctaacagctctatgaaccagcatctctcattgttccgtctctttgaactagaaagactgagattaaaggtctttatactcacttgaatatctttttatttttttcttttatattacctttttctagtgcatgagattcagccatcaaccgtgatgaactggatcatggaatctaacaatatcaagtttggatagaaaggagcttggcaatcttgtggtccatgcctcctcaatttaccaaggaagctgtgcaagctcaggtctccaggttcatcccagagaccccaccctggggttgcccgcagcattcctgtccttagaccccaaaaacaggctgctgggccctgtatttagggacctctgcactggctcttcttctctgggtgtatttctctgcacagagtgaaaagctgtgatgcacacagccagtcttcccacttctaatctgttctctgggctcccaggaacccaagcctctttctcaaacagccaaatgcccactgcccaggcctgcctgggcctccttccagatcaaggccagtttgcacaaccttaggcccaagggacagttgtttgcaaggggtgtgcactgagtagattgatgtttgcactggtgtttccaccgacgtttaaaggaggatgttcatgctggggcacagaacccaggtggaaagggaagaactgtgggtcagactttaattggtgtttcccacccaggaatacatattttaggggcaggtctgggcatggcacagctgtctgctatctctgcctcacttactctgcggaagtaaacaaacccccctgggttaacccaagtctagactgtgtccagcagctgtttcttgtgaggtagccctgagtagtgttgcttgtcttttgacctggtggctcaattggctaatatcccaaggtgcagaggacccccactgaggatgctgagtgggatggacctgtcctgcaggagcgggcgtgcttgccctctaatgcatttttgctttttgtaagaaaagaggacaaatgtctaatgtgcttttcagttcttttccaaattcagacaatagaggaaattagaggggattcaataaacccagaatattgttgtgtagcaaaatgagcatttccaacaaagaaactttgtgtgtttaaagaaagcaattctattttaaagcagcatgattgagccccacaaaacaaagtgtcagctacaagacaacaatcttttggtaaggtattggggcttacgatatctgttttgtgccctcttccttctggttcagagttgatctgctatgataaatattttatttggggagttttttggtactggacaataaacagggccacagctgattgccagagacaattttctaa

SEQ ID NO: 36 - DNA, 1397 bp, Homo sapiens
gcaactgtgatcctgagctgtgcacacttctggttgggattatttctggtttctacttcctgtttgaagatgtggcatggagagtgtaagttgaagagatgagtgcggggctcatctatccctggaattgtctttcccacaatccctgacacagaatatgagccatacaggaattctgaagaaatgggtctcttgccacctcccagtaaaagattattttttaaaaaaaaaagcacctggcatcacagccccagaactcagctatttccatgggaccacagcatctcatctctgggctgagccagaaagacccctactgaagtccagaggcacttttctgaaagggctctgctttgacctgaagtattttatctatcctcagtctcaggacactgttgatggaattaaagggcaaaagttttgcatccagaccatgcactctagcatcctaagtccatggctgctgtggcagtggctcttttcacaccaaaaatggagggagaaaaccagctaactcctgggctctctgactgcagtcaggccaaggaccagggaacctgcatctgttcactagggtgaggatttaaagcatcttctgggttttcagatcctaccaagaaaagcttcagaggcagaccttgagtctctgtgaaccccatcctgtattttaatgactgggctcctcagcagatctacagacttctagttcttttcggggtggtggtgactcttgacatttattgtccaggtctggtctgcagcacttaggatgcctctagcctatggagatggagttacacatcatgcatgttgactgtacatgctacaccacttctccagacttgacctctatcaaagggcatttgggtatggcccgagatgggagtaacctctcttttcagctgtgtcaaattcttacaccatgttcttttgttttttcctcataatacaaggccaagcacatctgcaaaaaagacattgctggaggaggtgcaaagagctggaaaccaagtctccagtcctgggaaaagcagtggtatggaaaagcaatggaaagagcattttgaaaatgccattccactgttttctggcctttatgatttctgctgagaaatccactgttagtctgatggggtctccttcatagcaccaatgacctgaagagccttgttgaaggaagactccatctgatgactcagagcaagtattttttagtgtgttattgttattagcagaaagagggccataaaatacatggggcaagctgaatatatcttaggcaaaagaagaaaatattcaaattcttatgttattttatctaattattttatctctttttgtgtgtgacttataatgtgtgtattgtattaataaaagtatataaacatgtagtttac

SEQ ID NO: 37 - DNA, 6509 bp, Homo sapiens
aatctcgctctgtcactcaggctggagtgcagtggcgcaatctcggctcactgcaaactctgcctcccaggttcacgccattctcctgccacagcctcccgagtagctgggactacaggcgcccgccaccaagcacagctaagttttttatttatagtagagacggggtttcactgtgttagccaggatggtctcgatctcctgacctcgtgatctgcctgcctcggcctcccaaagtgccgggattacaggcgtgagccaccgcgccgggcctgatttcagtttcttccagcccttcctattgttaacatgggggttgtgttgaagaatataaagttacaaagtcaaggaagtaggaaacatttttacaagtattatgtagccatcttggtggggctgtggtgaggtaggctgcaaatgattctcctatttctttccctgagttcagaacataggaattagattgatagacatcaacatacccgctttattgctgactcatgacaactaatgggaagacatggctcagatgtgcagccacagtgagcttctgaacatttcttctcagactaagctcttacacacagttgcagttgaaagaaagaattgcttgacatggccacaggagcaggcagcttcctgcagacatgacagtcaacgcaaactcatgtcactgtgggcagacacatgtttgcaaagagactcagagccaaacaagcacactcaatgtgctttgcccaaatttacccattaggtaaatcttccctcctcccaagaagaaagtggagagagcatgagtcctcacatggaaacttgaagtcagggaaatgaaggctcaccaattatttgtgcatgggtttaagttttccttgaaattaagttcaggtttgtctttgtgtgtaccaattaatgacaagaggttagatagaagtatgctagatggcaaagagaaatatgttttgtgtcttcaattttgctaaaaataacccagaacatggataattcatttattaattgattttggtaagccaagtcctatttggagaaaattaatagtttttctaaaaaagaattttctcaatatcacctggcttgataacatttttctccttcgagttcctttttctggagtttaacaaacttgttctttacaaatagattatattgactaccactcactgatgttatgatattagtttctattgcttactttgtatttctaattttaggattcacaatttagctggagaactattttttaacctgttgcacctaaacatgattgagctagaagacagttttaccatatgcatgcattttctctgagttatattttaaaatctatacatttctcctaaatatggaggaaatcactggcatcaaatgccagtctcagacggaagacctaaagcccatttctggcctggagctacttggctttgtgacctatggtgaggcataagtgctctgagtttgtgttgcctcttttgtaaaatgagggtttgacttaatcagtgattttcatagcttaaaatttttttgaagaacagaactttttttaaaaacagttagatgcaaccatattatataaaacagaacagatacaagtagagctaacttgctaaagaaaggatggaggctctgaagctgtgacttcattatcccttaatactgctatgtcctctgtagtaccttagatttctatgggacatcgtttaaaaactattgtttatgcgagagccttgctaatttcctaaaaattgtggatacattttttctcccatgtataattttctcaccttctatttaaaaagaaaaaaaaagtcagtgtagtatttacatattttaccctataaggagctaacataacttttgatttagtgttattcataaaattaggttagcagtttattaaccttttgtatttgctctggcaatgtttaatatctcataagctatacacacctcgaagccatcaatgacaaccttttcttgctgaatagaacagtgattgatgtcatgaagacaattttatctccttttgccttccataatttgtaccagcaggttatataatagtataacactgccaaggagcggattatctcatcttcatcctgtaattccagtgtttgtcacgtggttgttgaataaatgaataaagaatgagaaaaccagaagctctgatacataatcataatgataattatttcaatgcacaactacgggtggtgctgaactagaatctatattttctgaaactggctcctctaggatctactaatgatttaaatctaaaagatgaagttagtaaagcatcagaaaaaaaaggtgggtattcctacaagtcaggacattctacgtgactacaatataatctcacagaaatttaacattaatacattctaagatttaattctgtaaacaaattgctcctgtggagatgattggcatcacatggtgttttgagctgatacacccaacacttgagctcactgcaacagtaccagattttcaccgctatgcctcctttcactctgggagtcttccagaggtcttgcactcgggagagcatgctcaggtttccccagctctacaaaatcacccagaatgccaaagacttcaacacaagggtaaataaggttgatctcagaattgtcacctcaaaaaggccctgccttccactgttcagttctggtcatctgcctatgagatatctgaagcttgaaagagaacacttgaaaatcactgagaccgtgactcccatcccagcacacacagcaagccaagtaggttacagagatttcttcttgggtgatgagttcacgccacatggggtatgttctccagttccagtgtgttgactccttcctgcttccccccatcagcccctgaggtcaatgtgggcagcagcccccatggtccaagttcaagatccgctgtggaagacttttttaggcaatcacacagccctgcatggagtcctgatgagagcttgcctaattgttgctaggtttgtcattttaaatacagtgtttctttagctagtgagtaaaattggctatataggagatactgtgttgaccagtggtcatgccactgcctgttgatttgttgaaaatattgtttacacgtatgttcttgttactgattgtcagaaagctggttttgagactgcagcttggactaaattcagtcatctggctgtctggggaagcatgctgaccagtctggtgttctttggcatctactcagccatctggtccaccattctcattgccccaaatatgagaggacaggtaagtatgccagattggaagtttgccttctgtgtcttgtgataacaatgtttctccatgtgtaggcaagcaggctctctgtgttttacaactgtattgaggtttgatagacgctggtgctacctctttctgtcaggatctatcaggaggtaaaccaaattaaagcacctggaagatgtcatccaatccacagaaatggcatttcaaatttaaagtattataaaaagtcaacctaaatccccttacttacctggcatgtccacattttcataataaattatgtgttaattgcacatagtactgatatgtgcagtttatggccctttggaaactggagctattttgagttgctcataatccagtgattatactggttacagttttgatgttgtactctgattagtagttatcctctgctgagtcatctgtggaagttaaatctaacagctctatgaaccagcatctctcattgttccgtctctttgaactagaaagactgagattaaaggtctttatactcacttgaatatctttttatttttttcttttatattacctttttctagtgcatgagattcagccatcaaccgtgatgaactggatcatggaatctaacaatatcaagtttggatagaaaggagcttggcaatcttgtggtccatgcctcctcaatttaccaaggaagctgtgcaagctcaggtctccaggttcatcccagagaccccaccctggggttgcccgcagcattcctgtccttagaccccaaaaacaggctgctgggccctgtatttagggacctctgcactggctcttcttctctgggtgtatttctctgcacagagtgaaaagctgtgatgcacacagccagtcttcccacttctaatctgttctctgggctcccaggaacccaagcctctttctcaaacagccaaatgcccactgcccaggcctgcctgggcctccttccagatcaaggccagtttgcacaaccttaggcccaagggacagttgtttgcaaggggtgtgcactgagtagattgatgtttgcactggtgtttccaccgacgtttaaaggaggatgttcatgctggggcacagaacccaggtggaaagggaagaactgtgggtcagactttaattggtgtttcccacccaggaatacatattttaggggcaggtctgggcatggcacagctgtctgctatctctgcctcacttactctgcggaagtaaacaaacccccctgggttaacccaagtctagactgtgtccagcagctgtttcttgtgaggtagccctgagtagtgttgcttgtcttttgacctggtggctcaattggctaatatcccaaggtgcagaggacccccactgaggatgctgagtgggatggacctgtcctgcaggagcgggcgtgcttgccctctaatgcatttttgctttttgtaagaaaagaggacaaatgtctaatgtgcttttcagttcttttccaaattcagacaatagaggaaattagaggggattcaataaacccagaatattgttgtgtagcaaaatgagcatttccaacaaagaaactttgtgtgtttaaagaaagcaattctattttaaagcagcatgattgagccccacaaaacaaagtgtcagctacaagacaacaatcttttggtaaggtattggggcttacgatatctgttttgtgccctcttccttctggttcagagttgatctgctatgataaatattttatttggggagttttttggtactggacaataaacagggccacagctgattgccagagacaattttctaagcaactgtgatcctgagctgtgcacacttctggttgggattatttctggtttctacttcctgtttgaagatgtggcatggagagtgtaagttgaagagatgagtgcggggctcatctatccctggaattgtctttcccacaatccctgacacagaatatgagccatacaggaattctgaagaaatgggtctcttgccacctcccagtaaaagattattttttaaaaaaaaaagcacctggcatcacagccccagaactcagctatttccatgggaccacagcatctcatctctgggctgagccagaaagacccctactgaagtccagaggcacttttctgaaagggctctgctttgacctgaagtattttatctatcctcagtctcaggacactgttgatggaattaaagggcaaaagttttgcatccagaccatgcactctagcatcctaagtccatggctgctgtggcagtggctcttttcacaccaaaaatggagggagaaaaccagctaactcctgggctctctgactgcagtcaggccaaggaccagggaacctgcatctgttcactagggtgaggatttaaagcatcttctgggttttcagatcctaccaagaaaagcttcagaggcagaccttgagtctctgtgaaccccatcctgtattttaatgactgggctcctcagcagatctacagacttctagttcttttcggggtggtggtgactcttgacatttattgtccaggtctggtctgcagcacttaggatgcctctagcctatggagatggagttacacatcatgcatgttgactgtacatgctacaccacttctccagacttgacctctatcaaagggcatttgggtatggcccgagatgggagtaacctctcttttcagctgtgtcaaattcttacaccatgttcttttgttttttcctcataatacaaggccaagcacatctgcaaaaaagacattgctggaggaggtgcaaagagctggaaaccaagtctccagtcctgggaaaagcagtggtatggaaaagcaatggaaagagcattttgaaaatgccattccactgttttctggcctttatgatttctgctgagaaatccactgttagtctgatggggtctccttcatagcaccaatgacctgaagagccttgttgaaggaagactccatctgatgactcagagcaagtattttttagtgtgttattgttattagcagaaagagggccataaaatacatggggcaagctgaatatatcttaggcaaaagaagaaaatattcaaattcttatgttattttatctaattattttatctctttttgtgtgtgacttataatgtgtgtattgtattaataaaagtatataaacatgtagtttac

SEQ ID NO: 38 - DNA, 1397 bp, Homo sapiens; identical to SEQ ID NO: 36

SEQ ID NO: 39 - protein, 70 aa, Homo sapiens
NLALSLRLECSGAISAHCKLCLPGSRHSPATASRVAGTTGARHQAQLSFLFIVETGFHCVSQDGLDLLTS

SEQ ID NO: 40 - protein, 20 aa, Homo sapiens
SACLGLPKCRDYRREPPRRA

SEQ ID NO: 41 - protein, 42 aa, Homo sapiens
FQFLPALPIVNMGVVLKNIKLQSQGSRKHFYKYYVAILVGLW

SEQ ID NO: 42 - protein, 18 aa, Homo sapiens
GRLQMILLFLSLSSEHRN

SEQ ID NO: 43 - protein, 13 aa, Homo sapiens
QLMGRHGSDVQPQ

SEQ ID NO: 44 - protein, 8 aa, Homo sapiens
ASEHFFSD

SEQ ID NO: 45 - protein, 7 aa, Homo sapiens
ALTHSCS

SEQ ID NO: 46 - protein, 17 aa, Homo sapiens
KKELLDMATGAGSFLQT

SEQ ID NO: 47 - protein, 59 aa, Homo sapiens
QSTQTHVTVGRHMFAKRLRAKQAHSMCFAQIYPLGKSSLLPRRKWREHESSHGNLKSGK

SEQ ID NO: 48 - protein, 13 aa, Homo sapiens
RLTNYLCMGLSFP

SEQ ID NO: 49 - protein, 14 aa, Homo sapiens
VQVCLCVYQLMTRG

SEQ ID NO: 50 - protein, 4 aa, Homo sapiens
IEVC

SEQ ID NO: 51 - protein, 20 aa, Homo sapiens
MAKRNMFCVFNFAKNNPEHG

SEQ ID NO: 52 - protein, 12 aa, Homo sapiens
LILVSQVLFGEN

SEQ ID NO: 53 - protein, 30 aa, Homo sapiens
KRIFSISPGLITFFSFEFLFLEFNKLVLYK

SEQ ID NO: 54 - protein, 7 aa, Homo sapiens
IILTTTH

SEQ ID NO: 55 - protein, 47 aa, Homo sapiens
CYDISFYCLLCISNFRIHNLAGELFFNLLHLNMIELEDSFTICMHFL

SEQ ID NO: 56 - protein, 38 aa, Homo sapiens
NLYISPKYGGNHWHQMPVSDGRPKAHFWPGATWLCDLW

SEQ ID NO: 57 - protein, 16 aa, Homo sapiens
GISALSLCCLFCKMRV

SEQ ID NO: 58 - protein, 14 aa, Homo sapiens
LKIFLKNRTFFKNS

SEQ ID NO: 59 - protein, 12 aa, Homo sapiens
MQPYYIKQNRYK

SEQ ID NO: 60 - protein, 11 aa, Homo sapiens
LAKERMEALKL

SEQ ID NO: 61 - protein, 11 aa, Homo sapiens
LHYPLILLCPL

SEQ ID NO: 62 - protein, 9 aa, Homo sapiens
YLRFLWDIV

SEQ ID NO: 63 - protein, 9 aa, Homo sapiens
KLLFMREPC

SEQ ID NO: 64 - protein, 27 aa, Homo sapiens
FPKNCGYIFSPMYNFLTFYLKRKKKSV

SEQ ID NO: 65 - protein, 11 aa, Homo sapiens
YLHILPYKELT

SEQ ID NO: 66 - protein, 14 aa, Homo sapiens
VSSLLTFCICSGNV

SEQ ID NO: 67 - protein, 43 aa, Homo sapiens [sequence omitted]

SEQ ID NO: 68 - protein, 24 aa, Homo sapiens
YNTAKERIISSSSCNSSVCHVVVE

SEQ ID NO: 69 - protein, 4 aa, Homo sapiens
MNKE

SEQ ID NO: 70 - protein, 5 aa, Homo sapiens
ENQKL

SEQ ID NO: 71 - protein, 16 aa, Homo sapiens
YIIIMIIISMHNYGWC

SEQ ID NO: 72 - protein, 6 aa, Homo sapiens
TRIYIF

SEQ ID NO: 73 - protein, 4 aa, Homo sapiens
NWLL

SEQ ID NO: 74 - protein, 5 aa, Homo sapiens
DLLMI

SEQ ID NO: 75 - protein, 36 aa, Homo sapiens
KMKLVKHQKKKVGIPTSQDILRDYNIISQKFNINTF

SEQ ID NO: 76 - protein, 4 aa, Homo sapiens
DLIL

SEQ ID NO: 77 - protein, 14 aa, Homo sapiens
TNCSCGDDWHHMVF

SEQ ID NO: 78 - protein, 6 aa, Homo sapiens
ADTPNT

SEQ ID NO: 79 - protein, 48 aa, Homo sapiens
AHCNSTRFSPLCLLSLWESSRGLALGRACSGFPSSTKSPRMPKTSTQG

SEQ ID NO: 80 - protein, 24 aa, Homo sapiens
IRLISELSPQKGPAFHCSVLVICL

SEQ ID NO: 81 - protein, 6 aa, Homo sapiens
SLKENT

SEQ ID NO: 82 - protein, 5 aa, Homo sapiens
KSLRP

SEQ ID NO: 83 - protein, 10 aa, Homo sapiens
LPSQHTQQAK

SEQ ID NO: 84 - protein, 33 aa, Homo sapiens
VTEISSWVMSSRHMGYVLQFQCVDSFLLPPISP

SEQ ID NO: 85 - protein, 20 aa, Homo sapiens
GQCGQQPPWSKFKIRCGRLF

SEQ ID NO: 86 - protein, 9 aa, Homo sapiens
AITQPCMES

SEQ ID NO: 87 - protein, 7 aa, Homo sapiens
LLLGLSF

SEQ ID NO: 88 - protein, 6 aa, Homo sapiens
IQCFFS

SEQ ID NO: 89 - protein, 5 aa, Homo sapiens
VKLAI

SEQ ID NO: 90 - protein, 4 aa, Homo sapiens
EILC

SEQ ID NO: 91 - protein, 46 aa, Homo sapiens
PVVMPLPVDLLKILFTRMFLLLIVRKLVLRLQLGLNSVIWLSGEAC

SEQ ID NO: 92 - protein, 21 aa, Homo sapiens
PVWCSLASTQPSGPPFSLPQI

SEQ ID NO: 93 - protein, 31 aa, Homo sapiens
VCQIGSLPSVSCDNNVSPCVGKQALCVLQLY

SEQ ID NO: 94 - protein, 20 aa, Homo sapiens
GLIDAGATSFCQDLSGGKPN

SEQ ID NO: 95 - protein, 19 aa, Homo sapiens
STWKMSSNPQKWHFKFKVL

SEQ ID NO: 96 - protein, 24 aa, Homo sapiens
KVNLNPLTYLACPHFHNKLCVNCT

SEQ ID NO: 97 - protein, 13 aa, Homo sapiens
YVQFMALWKLELF

SEQ ID NO: 98 - protein, 18 aa, Homo sapiens
VAHNPVIILVTVLMLYSD

SEQ ID NO: 99 - protein, 39 aa, Homo sapiens
LSSAESSVEVKSNSSMNQHLSLFRLFELERLRLKVFILT

SEQ ID NO: 100 - protein, 35 aa, Homo sapiens
ISFYFFLLYYLFLVHEIQPSTVMNWIMESNNIKFG

SEQ ID NO: 101 - protein, 52 aa, Homo sapiens
KGAWQSCGPCLLNLPRKLCKLRSPGSSQRPHPGVARSIPVLRPQKQAAGPCI

SEQ ID NO: 102 - protein, 29 aa, Homo sapiens
GPLHWLFFSGCISLHRVKSCDAHSQSSHF

SEQ ID NO: 103 - protein, 50 aa, Homo sapiens
SVLWAPRNPSLFLKQPNAHCPGLPGPPSRSRPVCTTLGPRDSCLQGVCTE

SEQ ID NO: 104 - protein, 11 aa, Homo sapiens
IDVCTGVSTDV

SEQ ID NO: 105 - protein, 10 aa, Homo sapiens
LVFPTQEYIF

SEQ ID NO: 106 - protein, 26 aa, Homo sapiens
GQVWAWHSCLLSLPHLLCGSKQTPLG

SEQ ID NO: 107 - protein, 39 aa, Homo sapiens
PKSRLCPAAVSCEVALSSVACLLTWWLNWLISQGAEDPH

SEQ ID NO: 108 - protein, 14 aa, Homo sapiens
VGWTCPAGAGVLAL

SEQ ID NO: 109 - protein, 28 aa, Homo sapiens
CIFAFCKKRGQMSNVLFSSFPNSDNRGN

SEQ ID NO: 110 - protein, 24 aa, Homo sapiens
RGFNKPRILLCSKMSISNKETLCV

SEQ ID NO: 111 - protein, 9 aa, Homo sapiens
RKQFYFKAA

SEQ ID NO: 112 - protein, 16 aa, Homo sapiens
LSPTKQSVSYKTTIFW

SEQ ID NO: 113 - protein, 21 aa, Homo sapiens
GIGAYDICFVPSSFWFRVDLL

SEQ ID NO: 114 - protein, 17 aa, Homo sapiens
IFYLGSFLVLDNKQGHS

SEQ ID NO: 115 - protein, 6 aa, Homo sapiens
LPETIF

SEQ ID NO: 116 - protein, 33 aa, Homo sapiens
ATVILSCAHFWLGLFLVSTSCLKMWHGECKLKR

SEQ ID NO: 117 - protein, 65 aa, Homo sapiens [sequence omitted]

SEQ ID NO: 118 - protein, 18 aa, Homo sapiens
ARKTPTEVQRHFSERALL

SEQ ID NO: 119 - protein, 13 aa, Homo sapiens
PEVFYLSSVSGHC

SEQ ID NO: 120 - protein, 11 aa, Homo sapiens
RAKVLHPDHAL

SEQ ID NO: 121 - protein, 44 aa, Homo sapiens
HPKSMAAVAVALFTPKMEGENQLTPGLSDCSQAKDQGTCICSLG

SEQ ID NO: 122 - protein, 20 aa, Homo sapiens
GFKASSGFSDPTKKSFRGRP

SEQ ID NO: 123 - protein, 41 aa, Homo sapiens
VSVNPILYFNDWAPQQIYRLLVLFGVVVTLDIYCPGLVCST

SEQ ID NO: 124 - protein, 25 aa, Homo sapiens
DASSLWRWSYTSCMLTVHATPLLQT

SEQ ID NO: 125 - protein, 87 aa, Homo sapiens
PLSKGIWVWPEMGVTSLFSCVKFLHHVLLFFPHNTRPSTSAKKTLLEEVQRAGNQVSSPGKSSGMEKQWKEHFENAIPLFSGLYDFC

SEQ ID NO: 126 - protein, 4 aa, Homo sapiens
EIHC

SEQ ID NO: 127 - protein, 11 aa, Homo sapiens
SDGVSFIAPMT

SEQ ID NO: 128 - protein, 9 aa, Homo sapiens
RALLKEDSI

SEQ ID NO: 129 - protein, 6 aa, Homo sapiens
LRASIF

SEQ ID NO: 130 - protein, 43 aa, Homo sapiens
CVIVISRKRAIKYMGQAEYILGKRRKYSNSYVILSNYFISFCV

SEQ ID NO: 131 - protein, 7 aa, Homo sapiens
LIMCVLY

SEQ ID NO: 132 - protein, 5 aa, Homo sapiens
KYINM

SEQ ID NO: 133 - protein, 34 aa, Homo sapiens
ISLCHSGWSAVAQSRLTANSASQVHAILLPQPPE

SEQ ID NO: 134 - protein, 11 aa, Homo sapiens
LGLQAPATKHS

SEQ ID NO: 135 - protein, 4 aa, Homo sapiens
VFYL

SEQ ID NO: 136 - protein, 14 aa, Homo sapiens
RRGFTVLARMVSIS

SEQ ID NO: 137 - protein, 39 aa, Homo sapiens
PRDLPASASQSAGITGVSHRAGPDFSFFQPFLLLTWGLC

SEQ ID NO: 138 - protein, 15 aa, Homo sapiens
SYKVKEVGNIFTSIM

SEQ ID NO: 139 - protein, 12 aa, Homo sapiens
PSWWGCGEVGCK

SEQ ID NO: 140 - protein, 6 aa, Homo sapiens
FSYFFP

SEQ ID NO: 141 - protein, 22 aa, Homo sapiens
VQNIGIRLIDINIPALLLTHDN

SEQ ID NO: 142 - protein, 20 aa, Homo sapiens
VNLPSSQEESGESMSPHMET

SEQ ID NO: 143 - protein, 14 aa, Homo sapiens
SQGNEGSPIICAWV

SEQ ID NO: 144 - protein, 15 aa, Homo sapiens
VFLEIKFRFVFVCTN

SEQ ID NO: 145 - protein, 4 aa, Homo sapiens
QEVR

SEQ ID NO: 146 - protein, 29 aa, Homo sapiens
KYARWQREICFVSSILLKITQNMDNSFIN

SEQ ID NO: 147 - protein, 22 aa, Homo sapiens
AKSYLEKINSFSKKEFSQYHLA

SEQ ID NO: 148 - protein, 22 aa, Homo sapiens
HFSPSSSFFWSLTNLFFTNRLY

SEQ ID NO: 149 - protein, 24 aa, Homo sapiens
LPLTDVMILVSIAYFVFLILGFTI

SEQ ID NO: 150 - protein, 10 aa, Homo sapiens
LENYFLTCCT

SEQ ID NO: 151 - protein, 56 aa, Homo sapiens
KTVLPYACIFSELYFKIYTFLLNMEEITGIKCQSQTEDLKPISGLELLGFVTYGEA

SEQ ID NO: 152 - protein, 8 aa, Homo sapiens
VCVASFVK

SEQ ID NO: 153 - protein, 14 aa, Homo sapiens
GFDLISDFHSLKFF

SEQ ID NO: 154 - protein, 15 aa, Homo sapiens
RTELFLKTVRCNHII

SEQ ID NO: 155 - protein, 17 aa, Homo sapiens
NRTDTSRANLLKKGWRL

SEQ ID NO: 156 - protein, 7 aa, Homo sapiens
SCDFIIP

SEQ ID NO: 157 - protein, 46 aa, Homo sapiens
YCYVLCSTLDFYGTSFKNYCLCESLANFLKIVDTFFLPCIIFSPSI

SEQ ID NO: 158 - protein, 17 aa, Homo sapiens
KEKKSQCSIYIFYPIRS

SEQ ID NO: 159 - protein, 12 aa, Homo sapiens
FSVIHKIRLAVY

SEQ ID NO: 160 - protein, 12 aa, Homo sapiens
PFVFALAMFNIS

SEQ ID NO: 161 - protein, 14 aa, Homo sapiens
AIHTSKPSMTTFSC

SEQ ID NO: 162 - protein, 44 aa, Homo sapiens [sequence omitted]

SEQ ID NO: 163 - protein, 11 aa, Homo sapiens
IKNEKTRSSDT

SEQ ID NO: 164 - protein, 25 aa, Homo sapiens
LFQCTTTGGAELESIFSETGSSRIY

SEQ ID NO: 165 - protein, 5 aa, Homo sapiens
FKSKR

SEQ ID NO: 166 - protein, 20 aa, Homo sapiens
SIRKKRWVFLQVRTFYVTTI

SEQ ID NO: 167 - protein, 12 aa, Homo sapiens
SHRNLTLIHSKI

SEQ ID NO: 168 - protein, 74 aa, Homo sapiens [sequence omitted]

SEQ ID NO: 169 - protein, 26 aa, Homo sapiens
SQNCHLKKALPSTVQFWSSAYEISEA

SEQ ID NO: 170 - protein, 7 aa, Homo sapiens
KRTLENH

SEQ ID NO: 171 - protein, 21 aa, Homo sapiens
DRDSHPSTHSKPSRLQRFLLG

SEQ ID NO: 172 - protein, 63 aa, Homo sapiens
VHATWGMFSSSSVLTPSCFPPSAPEVNVGSSPHGPSSRSAVEDFFRQSHSPAWSPDESLPNCC

SEQ ID NO: 173 - protein, 13 aa, Homo sapiens
VCHFKYSVSLASE

SEQ ID NO: 174 - protein, 20 aa, Homo sapiens
NWLYRRYCVDQWSCHCLLIC

SEQ ID NO: 175 - protein, 10 aa, Homo sapiens
KYCLHVCSCY

SEQ ID NO: 176 - protein, 6 aa, Homo sapiens
LSESWF

SEQ ID NO: 177 - protein, 5 aa, Homo sapiens
DCSLD

SEQ ID NO: 178 - protein, 57 aa, Homo sapiens [sequence omitted]

SEQ ID NO: 179 - protein, 13 aa, Homo sapiens
ASRLSVFYNCIEV

SEQ ID NO: 180 - protein, 41 aa, Homo sapiens
TLVLPLSVRIYQEVNQIKAPGRCHPIHRNGISNLKYYKKST

SEQ ID NO: 181 - protein, 42 aa, Homo sapiens
IPLLTWHVHIFIINYVLIAHSTDMCSLWPFGNWSYFELLIIQ

SEQ ID NO: 182 - protein, 6 aa, Homo sapiens
LYWLQF

SEQ ID NO: 183 - protein, 22 aa, Homo sapiens
CCTLISSYPLLSHLWKLNLTAL

SEQ ID NO: 184 - protein, 11 aa, Homo sapiens
TSISHCSVSLN

SEQ ID NO: 185 - protein, 19 aa, Homo sapiens
RSLYSLEYLFIFFFYITFF

SEQ ID NO: 186 - protein, 8 aa, Homo sapiens
CMRFSHQP

SEQ ID NO: 187 - protein, 82 aa, Homo sapiens
TGSWNLTISSLDRKELGNLVVHASSIYQGSCASSGLQVHPRDPTLGLPAAFLSLDPKNRLLGPVFRDLCTGSSSLGVFLCTE

SEQ ID NO: 188 - protein, 49 aa, Homo sapiens
KAVMHTASLPTSNLFSGLPGTQASFSNSQMPTAQACLGLLPDQGQFAQP

SEQ ID NO: 189 - protein, 99 aa, Homo sapiens
AQGTVVCKGCALSRLMFALVFPPTFKGGCSCWGTEPRWKGKNCGSDFNWCFPPRNTYFRGRSGHGTAVCYLCLTYSAEVNKPPWVNPSLDCVQQLFLVR

SEQ ID NO: 190 - protein, 6 aa, Homo sapiens
VVLLVF

SEQ ID NO: 191 - protein, 6 aa, Homo sapiens
PGGSIG

SEQ ID NO: 192 - protein, 70 aa, Homo sapiens
YPKVQRTPTEDAEWDGPVLQERACLPSNAFLLFVRKEDKCLMCFSVLFQIQTIEEIRGDSINPEYCCVAK

SEQ ID NO: 193 - protein, 21 aa, Homo sapiens
AFPTKKLCVFKESNSILKQHD

SEQ ID NO: 194 - protein, 66 aa, Homo sapiens [sequence omitted]

SEQ ID NO: 195 - protein, 16 aa, Homo sapiens
AVHTSGWDYFWFLLPV

SEQ ID NO: 196 - protein, 8 aa, Homo sapiens
RCGMESVS

SEQ ID NO: 197 - protein, 139 aa, Homo sapiens [sequence omitted]

SEQ ID NO: 198 - protein, 19 aa, Homo sapiens
LLGSLTAVRPRTREPASVH

SEQ ID NO: 199 - protein, 25 aa, Homo sapiens
GEDLKHLLGFQILPRKASEADLESL

SEQ ID NO: 200 - protein, 17 aa, Homo sapiens
TPSCILMTGLLSRSTDF

SEQ ID NO: 201 - protein, 6 aa, Homo sapiens
FFSGWW

SEQ ID NO: 202 - protein, 28 aa, Homo sapiens
LLTFIVQVWSAALRMPLAYGDGVTHHAC

SEQ ID NO: 203 - protein, 24 aa, Homo sapiens
LYMLHHFSRLDLYQRAFGYGPRWE

SEQ ID NO: 204 - protein, 85 aa, Homo sapiens
PLFSAVSNSYTMFFCFFLIIQGQAHLQKRHCWRRCKELETKSPVLGKAVVWKSNGKSILKMPFHCFLAFMISAEKSTVSLMGSPS

SEQ ID NO: 205 - protein, 5 aa, Homo sapiens
PEEPC

SEQ ID NO: 206 - protein, 24 aa, Homo sapiens
RKTPSDDSEQVFFSVLLLLAERGP

SEQ ID NO: 207 - protein, 9 aa, Homo sapiens
NTWGKLNIS

SEQ ID NO: 208 - protein, 24 aa, Homo sapiens
AKEENIQILMLFYLIILSLFVCDL

SEQ ID NO: 209 - protein, 9 aa, Homo sapiens
CVYCINKSI

SEQ ID NO: 210 - protein, 5 aa, Homo sapiens
  misc_feature (5)..(5): Xaa can be any naturally occurring amino acid
TCSLX

SEQ ID NO: 211 - protein, 59 aa, Homo sapiens
SRSVTQAGVQWRNLGSLQTLPPRFTPFSCHSLPSSWDYRRPPPSTAKFFIYSRDGVSLC

SEQ ID NO: 212 - protein, 24 aa, Homo sapiens
PGWSRSPDLVICLPRPPKVPGLQA

SEQ ID NO: 213 - protein, 16 aa, Homo sapiens
ATAPGLISVSSSPSYC

SEQ ID NO: 214 - protein, 15 aa, Homo sapiens
HGGCVEEYKVTKSRK

SEQ ID NO: 215 - protein, 17 aa, Homo sapiens
ETFLQVLCSHLGGAVVR

SEQ ID NO: 216 - protein, 14 aa, Homo sapiens
AANDSPISFPEFRT

SEQ ID NO: 217 - protein, 8 aa, Homo sapiens
TSTYPLYC

SEQ ID NO: 218 - protein, 18 aa, Homo sapiens
LMTTNGKTWLRCAATVSF

SEQ ID NO: 219 - protein, 19 aa, Homo sapiens
TFLLRLSSYTQLQLKERIA

SEQ ID NO: 220 - protein, 47 aa, Homo sapiens
HGHRSRQLPADMTVNANSCHCGQTHVCKETQSQTSTLNVLCPNLPIR

SEQ ID NO: 221 - protein, 12 aa, Homo sapiens
IFPPPKKKVERA

SEQ ID NO: 222 - protein, 60 aa, Homo sapiens
VLTWKLEVREMKAHQLFVHGFKFSLKLSSGLSLCVPINDKRLDRSMLDGKEKYVLCLQFC

SEQ ID NO: 223 - protein, 49 aa, Homo sapiens
PRTWIIHLLIDFGKPSPIWRKLIVFLKKNFLNITWLDNIFLLRVPFSGV

SEQ ID NO: 224 - protein, 17 aa, Homo sapiens
QTCSLQIDYIDYHSLML

SEQ ID NO: 225 - protein, 8 aa, Homo sapiens
FLLLTLYF

SEQ ID NO: 226 - protein, 10 aa, Homo sapiens
DSQFSWRTIF

SEQ ID NO: 227 - protein, 7 aa, Homo sapiens
PVAPKHD

SEQ ID NO: 228 - protein, 23 aa, Homo sapiens
ARRQFYHMHAFSLSYILKSIHFS

SEQ ID NO: 229 - protein, 16 aa, Homo sapiens
IWRKSLASNASLRRKT

SEQ ID NO: 230 - protein, 11 aa, Homo sapiens
SPFLAWSYLAL

SEQ ID NO: 231 - protein, 15 aa, Homo sapiens
PMVRHKCSEFVLPLL

SEQ ID NO: 232 - protein, 5 aa, Homo sapiens
NEGLT

SEQ ID NO: 233 - protein, 6 aa, Homo sapiens
SVIFIA

SEQ ID NO: 234 - protein, 9 aa, Homo sapiens
NFFEEQNFF

SEQ ID NO: 235 - protein, 20 aa, Homo sapiens
KQLDATILYKTEQIQVELTC

SEQ ID NO: 236 - protein, 23 aa, Homo sapiens
RKDGGSEAVTSLSLNTAMSSVVP

SEQ ID NO: 237 - protein, 19 aa, Homo sapiens
ISMGHRLKTIVYARALLIS

SEQ ID NO: 238 - protein, 10 aa, Homo sapiens
KLWIHFFSHV

SEQ ID NO: 239 - protein, 21 aa, Homo sapiens
FSHLLFKKKKKVSVVFTYFTL

SEQ ID NO: 240 - protein, 15 aa, Homo sapiens
GANITFDLVLFIKLG

SEQ ID NO: 241 - protein, 25 aa, Homo sapiens
QFINLLYLLWQCLISHKLYTPRSHQ

SEQ ID NO: 242 - protein, 6 aa, Homo sapiens
QPFLAE

SEQ ID NO: 243 - protein, 12 aa, Homo sapiens
CHEDNFISFCLP

SEQ ID NO: 244 - protein, 9 aa, Homo sapiens
FVPAGYIIV

SEQ ID NO: 245 - protein, 12 aa, Homo sapiens
HCQGADYLIFIL

SEQ ID NO: 246 - protein, 8 aa, Homo sapiens
FQCLSRGC

SEQ ID NO: 247 - protein, 26 aa, Homo sapiens
RMRKPEALIHNHNDNYFNAQLRVVLN

SEQ ID NO: 248 - protein, 40 aa, Homo sapiens
NLYFLKLAPLGSTNDLNLKDEVSKASEKKGGYSYKSGHST

SEQ ID NO: 249 - protein, 8 aa, Homo sapiens
LQYNLTEI

SEQ ID NO: 250 - protein, 15 aa, Homo sapiens
YILRFNSVNKLLLWR

SEQ ID NO: 251 - protein, 8 aa, Homo sapiens
LASHGVLS

SEQ ID NO: 252 - protein, 129 aa, Homo sapiens
YTQHLSSLQQYQIFTAMPPFTLGVFQRSCTRESMLRFPQLYKITQNAKDFNTRVNKVDLRIVTSKRPCLPLFSSGHLPMRYLKLEREHLKITETVTPIPAHTASQVGYRDFFLGDEFTPHGVCSPVPVC

SEQ ID NO: 253 - protein, 59 aa, Homo sapiens
LLPASPHQPLRSMWAAAPMVQVQDPLWKTFLGNHTALHGVLMRACLIVARFVILNTVFL

SEQ ID NO: 254 - protein, 21 aa, Homo sapiens
LVSKIGYIGDTVLTSGHATAC

SEQ ID NO: 255 - protein, 76 aa, Homo sapiens
FVENIVYTYVLVTDCQKAGFETAAWTKFSHLAVWGSMLTSLVFFGIYSAIWSTILIAPNMRGQVSMPDWKFAFCVL

SEQ ID NO: 256 - protein, 35 aa, Homo sapiens
QCFSMCRQAGSLCFTTVLRFDRRWCYLFLSGSIRR

SEQ ID NO: 257 - protein, 19 aa, Homo sapiens
TKLKHLEDVIQSTEMAFQI

SEQ ID NO: 258 - protein, 19 aa, Homo sapiens
SIIKSQPKSPYLPGMSTFS

SEQ ID NO: 259 - protein, 22 aa, Homo sapiens
LHIVLICAVYGPLETGAILSCS

SEQ ID NO: 260 - protein, 13 aa, Homo sapiens
SSDYTGYSFDVVL

SEQ ID NO: 261 - protein, 6 aa, Homo sapiens
LVVILC

SEQ ID NO: 262 - protein, 5 aa, Homo sapiens
VICGS

SEQ ID NO: 263 - protein, 13 aa, Homo sapiens
QLYEPASLIVPSL

SEQ ID NO: 264 - protein, 28 aa, Homo sapiens
TRKTEIKGLYTHLNIFLFFSFILPFSSA

SEQ ID NO: 265 - protein, 13 aa, Homo sapiens
DSAINRDELDHGI

SEQ ID NO: 266 - protein, 47 aa, Homo sapiens
QYQVWIERSLAILWSMPPQFTKEAVQAQVSRFIPETPPWGCPQHSCP

SEQ ID NO: 267 - protein, 31 aa, Homo sapiens
TPKTGCWALYLGTSALALLLWVYFSAQSEKL

SEQ ID NO: 268 - protein, 58 aa, Homo sapiens
CTQPVFPLLICSLGSQEPKPLSQTAKCPLPRPAWASFQIKASLHNLRPKGQLFARGVH

SEQ ID NO: 269 - protein, 63 aa, Homo sapiens
CLHWCFHRRLKEDVHAGAQNPGGKGRTVGQTLIGVSHPGIHILGAGLGMAQLSAISASLTLRK

SEQ ID NO: 270 - protein, 9 aa, Homo sapiens
TNPPGLTQV

SEQ ID NO: 271 - protein, 8 aa, Homo sapiens
TVSSSCFL

SEQ ID NO: 272 - protein, 4 aa, Homo sapiens
GSPE

SEQ ID NO: 273 - protein, 46 aa, Homo sapiens
CCLSFDLVAQLANIPRCRGPPLRMLSGMDLSCRSGRACPLMHFCFL

SEQ ID NO: 274 - protein, 6 aa, Homo sapiens
EKRTNV

SEQ ID NO: 275 - protein, 11 aa, Homo sapiens
CAFQFFSKFRQ

SEQ ID NO: 276 - protein, 7 aa, Homo sapiens
RKLEGIQ

SEQ ID NO: 277 - protein, 6 aa, Homo sapiens
TQNIVV

SEQ ID NO: 278 - protein, 19 aa, Homo sapiens
QNEHFQQRNFVCLKKAILF

SEQ ID NO: 279 - protein, 38 aa, Homo sapiens
SSMIEPHKTKCQLQDNNLLVRYWGLRYLFCALFLLVQS

SEQ ID NO: 280 - protein, 16 aa, Homo sapiens
SAMINILFGEFFGTGQ

SEQ ID NO: 281 - protein, 41 aa, Homo sapiens
TGPQLIARDNFLSNCDPELCTLLVGIISGFYFLFEDVAWRV

SEQ ID NO: 282 - protein, 19 aa, Homo sapiens
VEEMSAGLIYPWNCLSHNP

SEQ ID NO: 283 - protein, 5 aa, Homo sapiens
AIQEF

SEQ ID NO: 284 - protein, 9 aa, Homo sapiens
RNGSLATSQ

SEQ ID NO: 285 - protein, 4 aa, Homo sapiens
KIIF

SEQ ID NO: 286 - protein, 31 aa, Homo sapiens [sequence omitted]

SEQ ID NO: 287 - protein, 6 aa, Homo sapiens
SPEALF

SEQ ID NO: 288 - protein, 6 aa, Homo sapiens
KGSALT

SEQ ID NO: 289 - protein, 29 aa, Homo sapiens [sequence omitted]

SEQ ID NO: 290 - protein, 25 aa, Homo sapiens
VHGCCGSGSFHTKNGGRKPANSWAL

SEQ ID NO: 291 - protein, 18 aa, Homo sapiens
LQSGQGPGNLHLFTRVRI

SEQ ID NO: 292 - protein, 27 aa, Homo sapiens
SIFWVFRSYQEKLQRQTLSLCEPHPVF

SEQ ID NO: 293 - protein, 19 aa, Homo sapiens
LGSSADLQTSSSFRGGGDS

SEQ ID NO: 294 - protein, 14 aa, Homo sapiens
HLLSRSGLQHLGCL

SEQ ID NO: 295 - protein, 55 aa, Homo sapiens
PMEMELHIMHVDCTCYTTSPDLTSIKGHLGMARDGSNLSFQLCQILTPCSFVFSS

SEQ ID NO: 296 - protein, 39 aa, Homo sapiens
YKAKHICKKDIAGGGAKSWKPSLQSWEKQWYGKAMERAF

SEQ ID NO: 297 - protein, 10 aa, Homo sapiens
KCHSTVFWPL

SEQ ID NO: 298 - protein, 9 aa, Homo sapiens
FLLRNPLLV

SEQ ID NO: 299 - protein, 33 aa, Homo sapiens
WGLLHSTNDLKSLVEGRLHLMTQSKYFLVCYCY

SEQ ID NO: 300 - protein, 11 aa, Homo sapiens
QKEGHKIHGAS

SEQ ID NO: 301 - protein, 17 aa, Homo sapiens
IYLRQKKKIFKFLCYFI

SEQ ID NO: 302 - protein, 26 aa, Homo sapiens
  misc_feature (26)..(26): Xaa can be any naturally occurring amino acid
LFYLFLCVTYNVCIVLIKVYKHVVYX

SEQ ID NO: 303 - protein, 80 aa, Homo sapiens
WEDMAQMCSHSELLNISSQTKLLHTVAVERKNCLTWPQEQAASCRHDSQRKLMSLWADTCLQRDSEPNKHTQCALPKFTH

(19) United States
(12) Patent Application Publication
(10) Pub. No.: US 2011/0052593 A1
(43) Pub. Date: Mar. 3, 2011

(54) BREAST CANCER ASSOCIATED ANTIGEN

(76) Inventors: Robert Charles Rees, Sheffield (GB); Stephanie A.S. Laversin, (US)

(21) Appl. No.: 12/738,211

(22) PCT Filed: Oct. 15, 2008
(86) PCT No.: PCT/GB2008/003492

§ 371 Date: Sep. 15, 2010

(30) Foreign Application Priority Data
Oct. 15, 2007 (GB) 0720156.9

(51) Int. Cl. A61K 39/395; C07H 21/04; C12N 15/63; C12N 5/10; C07K 2/00; C07K 7/06; C07K 16/18; C12Q 1/68; G01N 33/53; A61K 31/711; A61K 38/00; A61K 38/08; A61P 35/00

(52) U.S. Cl. 424/141.1; 536/23.5; 435/320.1; 435/325; 530/300; 530/328; 530/391.1; 435/6; 435/7.92; 514/44 R; 514/19.3; 514/19.4

(57) ABSTRACT

The invention relates to nucleic acid and amino acid sequences encoding BUC11 (SEQ ID NO: 36), BUC6 (SEQ ID NO: 31), BUC9 (SEQ ID NO: 32), and BUC10 (SEQ ID NO: 35), and to the use of such sequences in the identification and treatment of cancers.

### CROSS-REFERENCE TO RELATED APPLICATIONS

This application is a U.S. national counterpart application, filed under 35 U.S.C. §371(c), of international application serial No. PCT/GB2008/003492 filed Oct. 15, 2008, which claims priority to Great Britain Patent Application No. 0720156.9 filed Oct. 15, 2007.

Breast cancer is the most prevalent malignancy affecting women world-wide. Each year, there are nearly 41,000 new cases in the UK. This cancer accounts for almost one in three of all cancer cases in women, and the lifetime risk for breast cancer in women is one in nine (1). The treatment options for patients with advanced breast cancer are limited and improved treatment modalities are desperately needed, both from the compassionate and socio-economic viewpoints. In breast cancer, in spite of the identification of the tumour suppressor genes BRCA1/2 (2) and HER2/neu (3), there is an urgent need to identify new diagnostic tools and methods for treating such cancers.

Accordingly, there is a need to identify additional novel breast cancer-associated genes and proteins, which may have important implications for understanding the process of tumourigenesis, diagnosis and prognosis, and development of novel treatment regimens. This also includes assays for such newly identified breast cancer-associated genes.

Identification of immunogenic proteins in cancer is essential for the development of immunotherapeutic strategies where adoptive immunity is directed towards MHC Class I- and Class II-associated peptides (Mians, et al., Cancer Immunology (2001), page 1). Many antigens are implicated in aetiology and progression of cancer, and are associated with epigenetic events. Pre-clinical and clinical studies infer that vaccination and targeting MHC-associated peptide antigens promotes tumour rejection (Ali S. A., et al, J. Immunol. (2002), Vol. 168(7), pages 3512-19 and Rees R. C., et al., Immunol. Immunother (2002), Vol 51(1), pages 58-61).

Expressed sequence tags (ESTs) are single-pass reads from randomly selected cDNA clones. ESTs belong to different cDNA libraries, each of which is prepared from one particular cell type, organ, or tumour. Therefore, the presence or absence of ESTs in different libraries provides information about the organ, cell type, or tumour specificity of expressed genes. Also, a gene is often presented by several ESTs; generally, the more a gene is expressed in a given tissue, the more ESTs for that gene will be found in the library. Thus, the number of ESTs that represent the same gene in a given library is a rough indication of the expression level of the gene in the tissue from which the library is derived. ESTs have proved to be a valuable resource for detection and characterisation of new candidate genes. ESTs also provide an ideal resource to predict alternative splicing variants by examining all the features in genomic-EST-mRNA multiple alignments (4).

Since the breast is not essential after the reproductive years, unintentional destruction of normal breast tissues by a given of novel therapeutic strategies, such as immune therapy and gene therapy, for patients with breast cancer should have minimal consequences.

In order to identify new breast-associated genes, the inventors developed a strategy to mine the UniGene database (http://www.ncbi.nlm.nih.gov/entrez/query.fcgi?db=unigene). The NCBI describes UniGene as an experimental system for automatically partitioning GenBank® (i.e. the US National Institutes of Health (NIH) genetic sequence database: an annotated collection of all publicly available DNA sequences) sequences into a non-redundant set of gene-oriented ‘clusters’. Each UniGene cluster contains sequences that represent a unique gene, as well as related information such as the tissue types in which the gene has been expressed and map location.

The inventors mined UniGene for transcripts expressed exclusively in human breast. A number of different sequences from the UniGene hits were then selected by the inventors. Subsequently, reverse transcription polymerase chain reaction (RT-PCR) was used to validate the expression patterns of the selected UniGene ‘clusters’. Such clusters are ESTs (Expressed Sequence Tags) considered to be derived from the same gene.

A first aspect of the present invention provides an isolated mammalian nucleic acid molecule selected from the group consisting of:

a) a nucleic acid sequence encoding BUC11 (SEQ ID NO: 36), BUC6 (SEQ ID NO: 31), BUC9 (SEQ ID NO: 32) and/or BUC10 (SEQ ID NO: 35);

b) a nucleic acid sequence comprising the nucleic acid sequence encoding at least one amino acid open reading frame obtainable from a nucleic acid sequence encoding BUC11 (SEQ ID NO: 36), BUC6 (SEQ ID NO: 31), BUC9 (SEQ ID NO: 32) and/or BUC10 (SEQ ID NO: 35), wherein a peptide having the amino acid sequence of the encoded reading frame is capable of cross-reacting with samples from patients susceptible to cancer and/or from samples from patients with cancer;

c) a fragment of a nucleic acid molecule as defined in a) or b) comprising at least 20 contiguous nucleotides or which encodes at least 4 amino acids of the sequence;

d) nucleic acid molecules, the complementary strand of which specifically hybridises to nucleic acid molecules described in a), b) or c);

e) nucleic acid molecules, the sequence of which differs from the sequence of the nucleic acid molecule of a), b) c) or d) due to the degeneracy of the genetic code;

f) nucleic acid molecules comprising at least 10 contiguous nucleotides capable of specifically hybridising to a nucleic acid molecule as defined in a), b), c), d) or e).

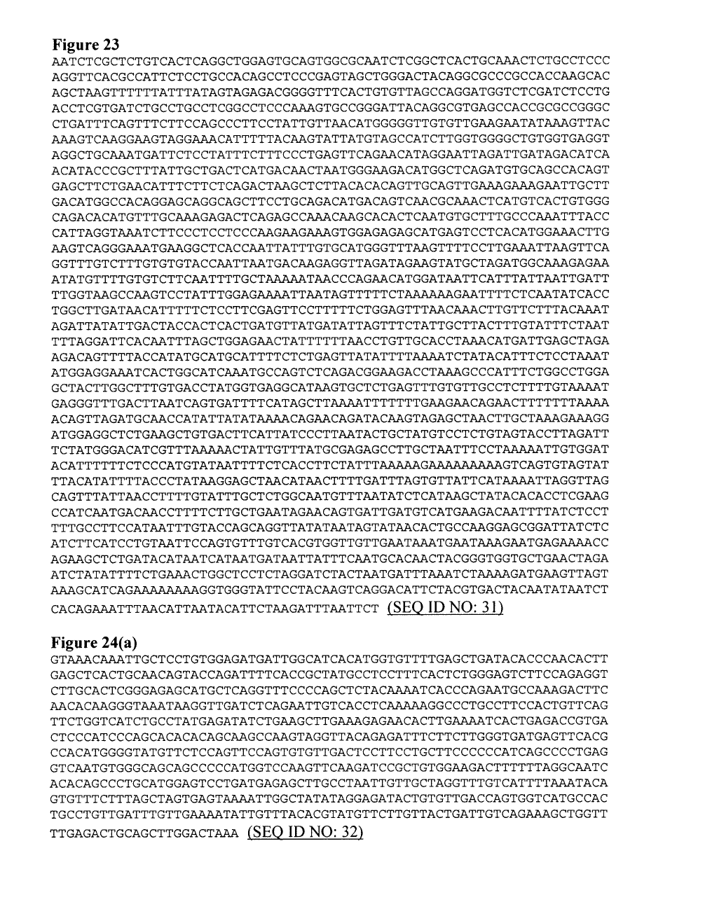

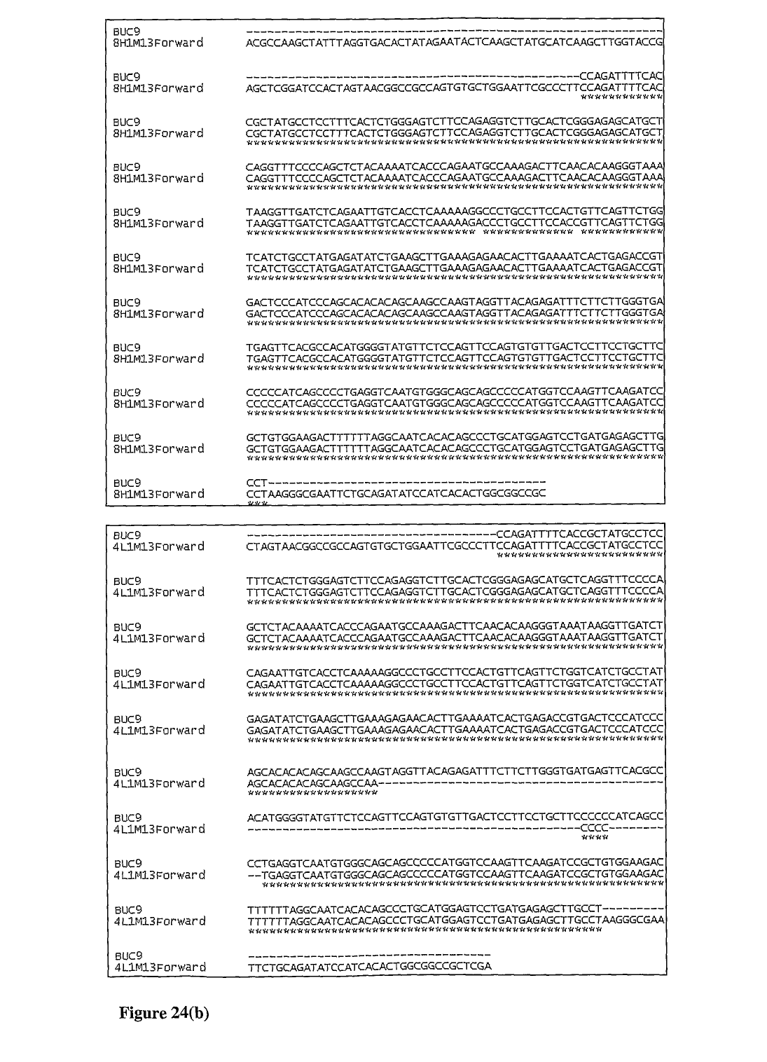

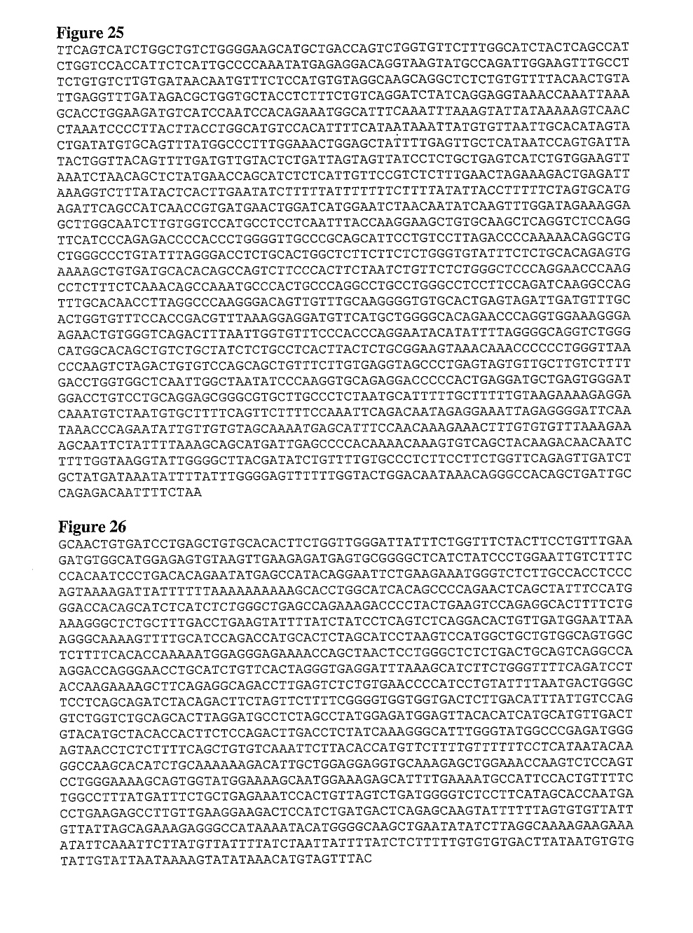

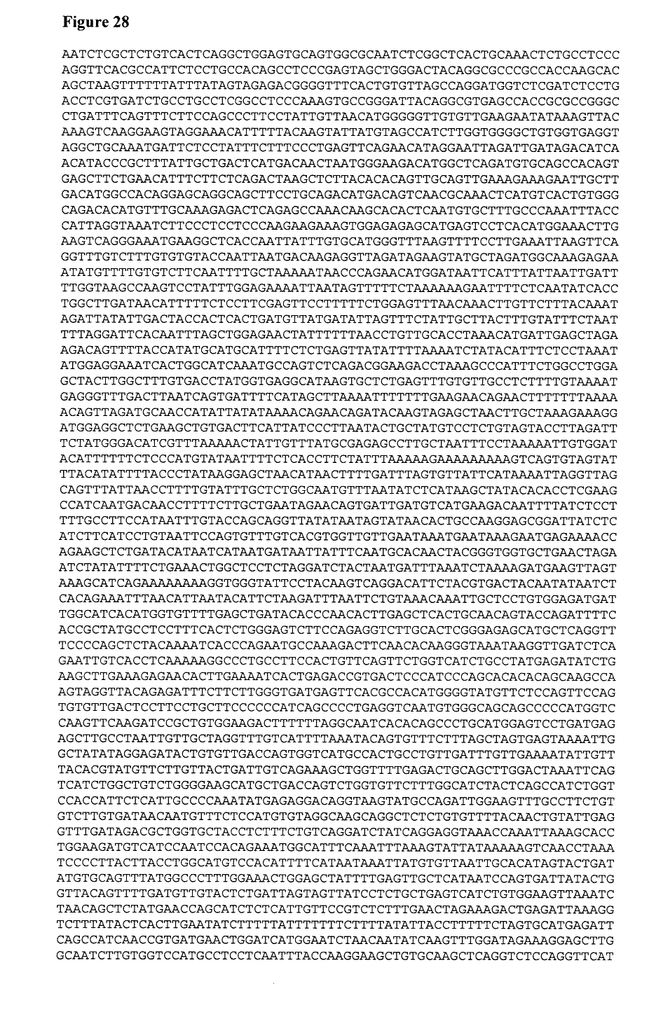

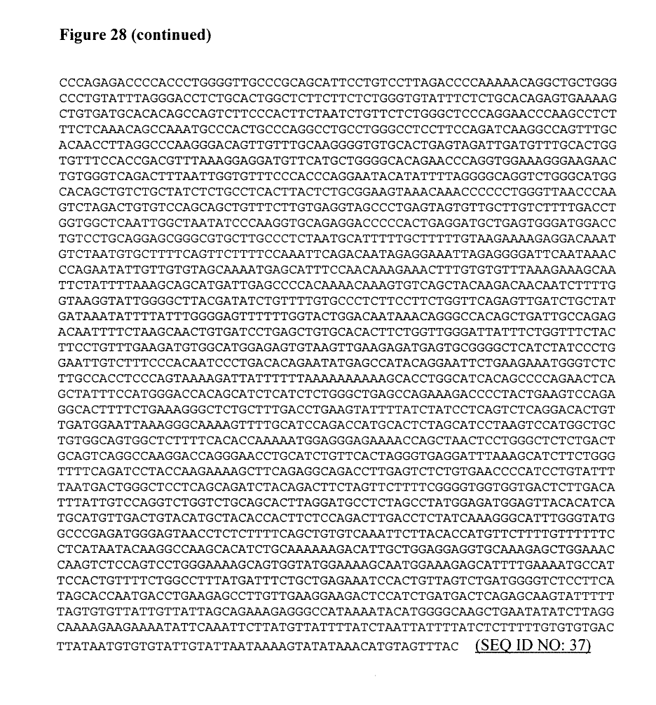

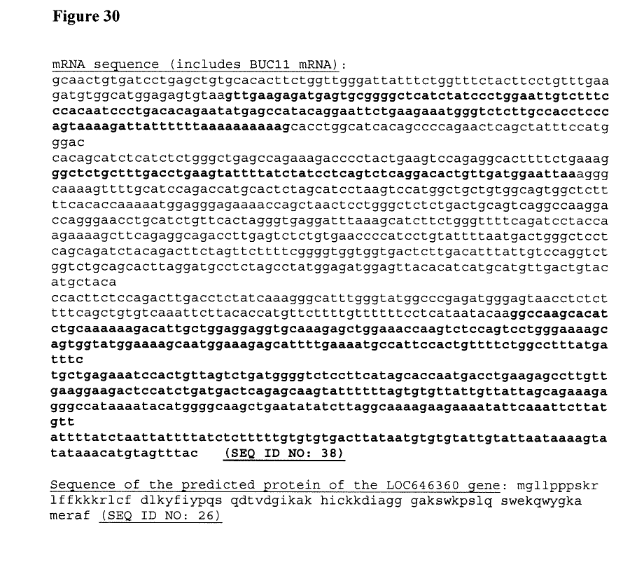

Preferably, the nucleic acid molecule a) comprises a sequence selected from a nucleic acid sequence shown in FIG. 26 (BUC11 (SEQ ID NO: 36)); FIG. 30 (BUC11 (SEQ ID NO: 36) mRNA including the mRNA sequence of LOC 646360); FIG. 23 (BUC6) (SEQ ID NO: 31), FIG. 24(a) (BUC9 (SEQ ID NO: 32)); FIG. 24(b) BUC9 (SEQ ID NO: 32) 8H1M13 or BUC9 (SEQ ID NO: 32) 4L1M13 splice variants; FIG. 25 (BUC10 (SEQ ID NO: 35)), or FIG. 28 (alignment for BUC6 (SEQ ID NO: 31), BUC9 (SEQ ID NO: 32), BUC10 (SEQ ID NO: 35) and BUC11 (SEQ ID NO: 36)).

Preferably the fragment of nucleic acid (c) encodes at least 20, at least 30, at least 40, at least 50, at least 60, at least 80 or at least 100 nucleotides of the sequences. Preferably the fragment encodes a peptide capable of cross reacting with sera from patients with cancer and/or of being specifically bound by an antibody against BUC11 (SEQ ID NO: 36), BUC6 (SEQ ID NO: 31), BUC 9 (SEQ ID NO: 32) or BUC10 (SEQ ID NO: 35).

The BUC11 (SEQ ID NO: 36) antibody may be one raised against the peptide:

PSKRLFFKKKRLC (SEQ ID NO: 1).

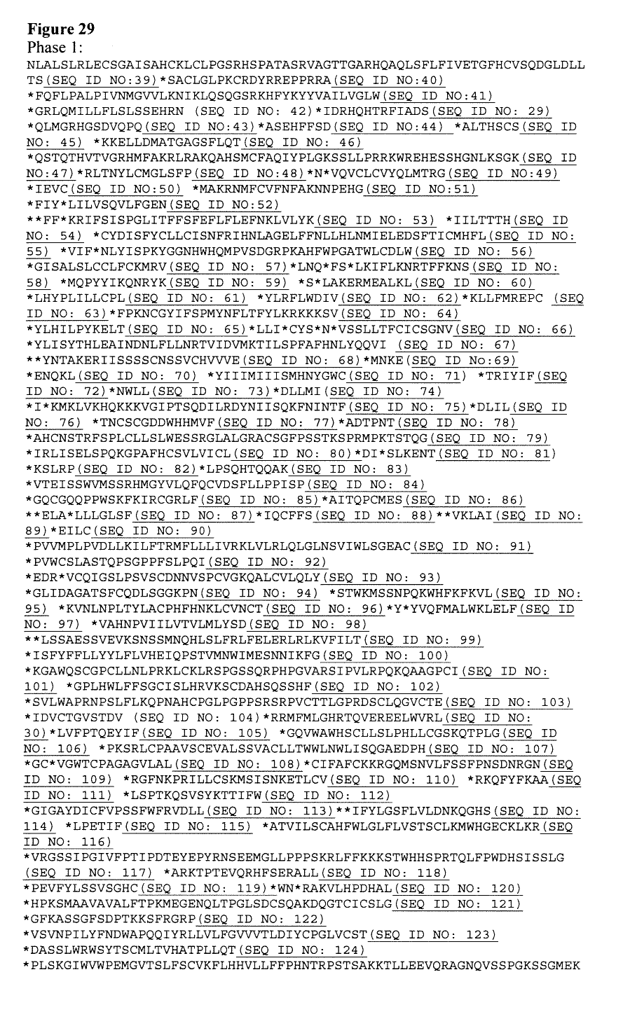

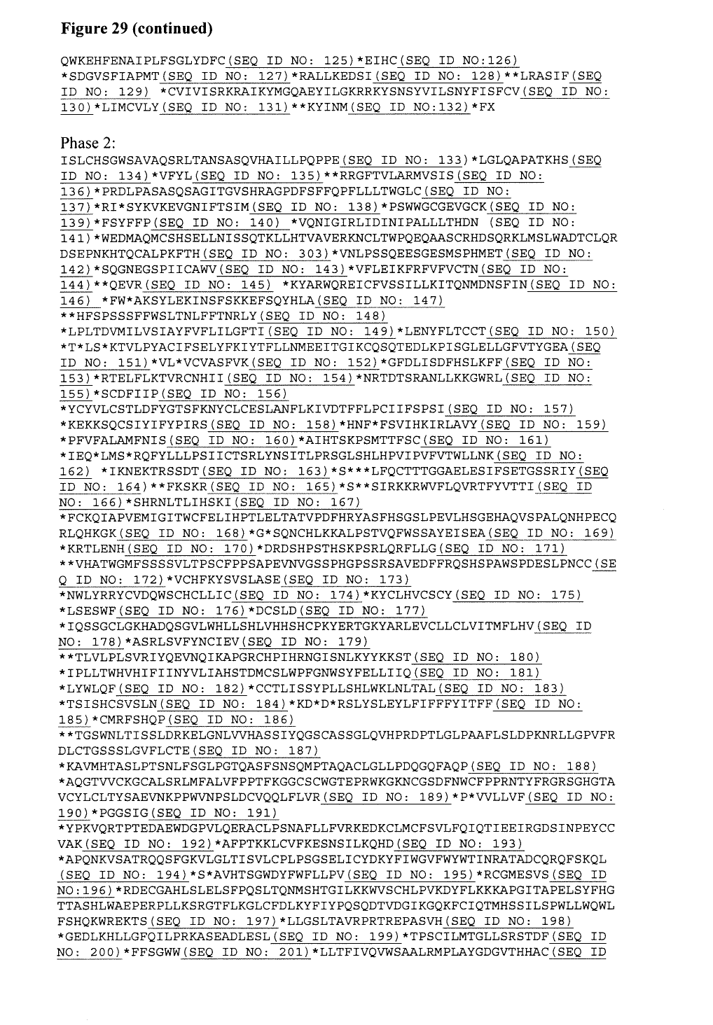

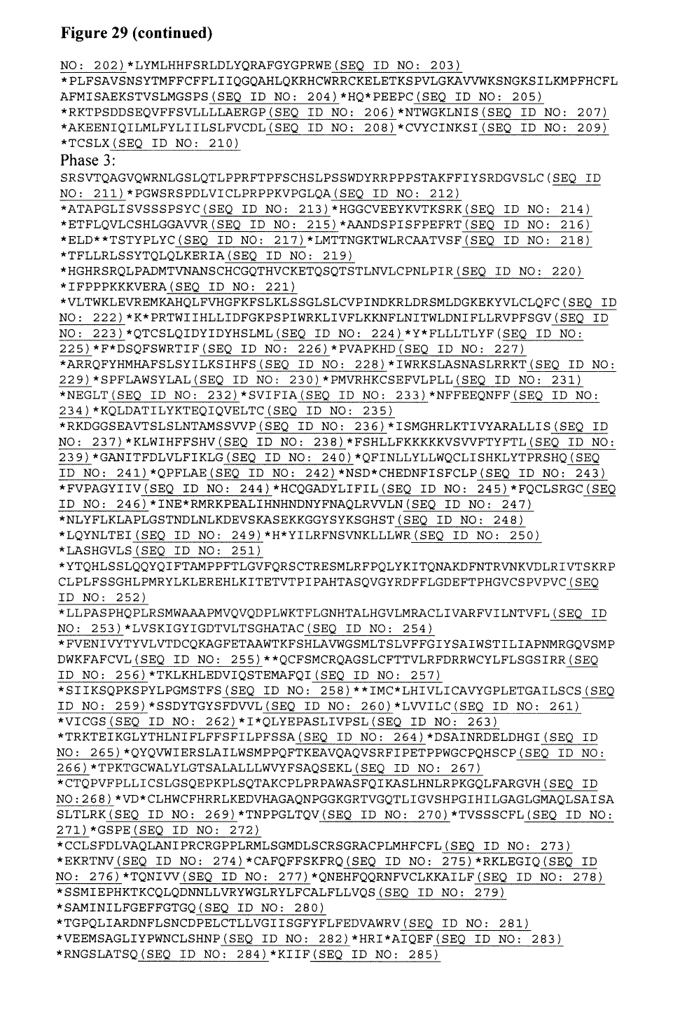

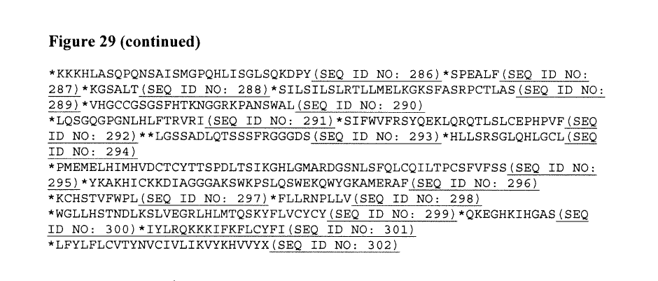

Additionally or alternatively the fragment encodes at least 10, at least 20, at least 30, most preferably all of the amino acids of the BUC6 (SEQ ID NO: 31) sequence of FIG. 29.

Preferably, the fragment (c) of the nucleic acid comprises at least 20 nucleotides from nt 54-1809 of the sequence shown in FIG. 23.

Preferably, the fragment does not encode a sequence found in the NY-BR-1 sequence, human DNA clone RP11-141F12, or LOC646360.

The claimed nucleic acid sequences are associated with cancer. The nucleic acid molecules and their products may be used in the diagnosis, prognosis and/or management of cancer. They may be used for targets for gene/immune therapy for breast cancer.

BUC11 (SEQ ID NO: 36) is expected to be useful in the management of testicular cancer and in the diagnosis/prognosis of breast cancer.

BUC6 (SEQ ID NO: 31), 9 (SEQ ID NO: 32) and 11 (SEQ ID NO: 36) are highly expressed in breast cancer tissues and are expected to be useful for the detection of metastatic breast cancer cells in axillary lymph nodes as well as the peripheral blood stream. Moreover, the splice variants of BUC9 (SEQ ID NO: 32) may be an indication of breast cancer disease state.

BUC6 (SEQ ID NO: 31), BUC9 (SEQ ID NO: 32) and BUC10 (SEQ ID NO: 35) are expressed in melanoma and are expected to be useful in management of melanoma.

BUC9 (SEQ ID NO: 32) and BUC10 (SEQ ID NO: 35) are expected to be useful in the management of oesophogeal cancer and gastric cancer.

The nucleic acid may or may not be translated into protein in, for example, a mammalian body, such as the human body. Preferably the nucleic acid is expressed as mRNA. Preferably the mRNA is expressed in higher than normal concentrations in cancer tissue compared to one or more of normal adrenal gland, brain, foetal brain, foetal liver, heart, kidney, liver, lung, placenta, prostate, saliva, skeletal muscle, spinal cord, spleen, thymus, thyroid, trachea and/or uterus. The mRNA is usually translated into protein.

BUC11 (SEQ ID NO: 36) is expressed in normal breast and normal testis. It is highly expressed in breast cancer tissues and testicular cancer tissues. It is not expressed in PBMC, mesothelioma tissues, melanoma tissues, gastric tissues, kidney tissues and oesophogeal tissues.

BUC6 (SEQ ID NO: 31) is expressed in breast cancer tissue. BUC6 (SEQ ID NO: 31) is also highly expressed in melanoma and normal breast tissues, testes and a breast cancer cell line T470.

BUC9 (SEQ ID NO: 32) is highly expressed in breast cancer cell lines and is expressed in normal placenta, testis and breast. It is also expressed in oesophogeal (paired normal and cancer) tissues and has been observed in 50% of the gastric (paired normal and cancer) tissues tested. BUC9 (SEQ ID NO: 32) has also been found to be highly expressed in melanoma.

BUC10 (SEQ ID NO: 35) is highly expressed in melanoma and breast cancer cell lines, but mRNA expression is lower in breast tissues than some normal tissues.

Nucleic acid molecules having at least 60%, 70%, 75%, 80%, 85%, 90%, 95%, 98% or 99% homology to the nucleic acid molecules are also provided. Preferably these nucleic acid molecules express proteins which are expressed in higher concentrations in cancerous tissue than the equivalent normal tissue. Preferably this is at least 2, more preferably at least 5, most preferably at least 10 times higher concentrations than normal tissue.

Preferably the nucleic acid molecule is itself expressed in higher concentrations in breast cancer tissue, and/or in secondary tumours derived from breast cancer tissue, than in normal non-cancerous breast tissue. BUC6 (SEQ ID NO: 31) has been found to be expressed in prostate cancer at approximately 1/10 the level of expression in breast cancer. It was not observed to be expressed in gastric cancer, oesophageal cancer, renal cancer or head or neck tumours.

The nucleic acid molecules of the invention may be DNA, cDNA or RNA. In RNA molecules “T” (Thymine) residues may be replaced by “U” (Uridine) residues.

Preferably, the isolated mammalian nucleic acid molecule is an isolated human nucleic acid molecule.

The invention further provides nucleic acid molecules comprising at least 15 nucleotides capable of specifically hybridising to a sequence included within the sequence of a nucleic acid molecule according to the first aspect of the invention. The hybridising nucleic acid molecule may either be DNA or RNA. Preferably the molecule is at least 90%, at least 92%, at least 94%, at least 95%, at least 96%, at least 98%, at least 99%, homologous to the nucleic acid molecule according to the first aspect of the invention. This may be determined by techniques known in the art.

The term “specifically hybridising” is intended to mean that the nucleic acid molecule can hybridise to nucleic acid molecules according to the invention under conditions of high stringency. Typical conditions for high stringency include 0.1×SET, 0.1% SDS at 68° C. for 20 minutes.

The invention also encompasses variant nucleic acid molecules such as DNAs and cDNAs which differ from the sequences identified above, but encode the same amino acid sequences as the isolated mammalian nucleic acid molecules, by virtue of redundancy in the genetic code.

siRNA encoding at least a portion of BUC11 (SEQ ID NO: 36), BUC6 (SEQ ID NO: 31), BUC9 (SEQ ID NO: 32) and BUC10 (SEQ ID NO: 35) nucleotide sequences is also provided by the invention.

| | | | |
|---|---|---|---|
| U | C | A | G |

| | | | | | | | | | | | | | |
|---|---|---|---|---|---|---|---|---|---|---|---|---|---|
| U | UUU | | | UCU | | | UAU | | | UGU | | | U |
| | | {close oversize bracket} | Phe | | | | | {close oversize bracket} | Tyr | | {close oversize bracket} | Cys | |
| | UUC | | | UCC | | | UAC | | | UGC | | | C |
| | UUA | | | UCA | {close oversize bracket} | Ser | UAA* | | Stop | UGA* | | Stop | A |
| | | {close oversize bracket} | Leu | | | | | | | | | | |
| | UUG | | | UCG | | | UAG* | | Stop | UGG | | Trp | G |
| C | CUU | | | CCU | | | CAU | | | CGU | | | U |
| | | | | | | | | {close oversize bracket} | His | | | | |
| | CUC | | | CCC | | | CAC | | | CGC | | | C |
| | CUA | {close oversize bracket} | Leu | CCA | {close oversize bracket} | Pro | CAA | | | CGA | {close oversize bracket} | Arg | A |
| | | | | | | | | {close oversize bracket} | Gln | | | | |
| | CUG | | | CCG | | | CAG | | | CGG | | | G |
| A | AUU | | | ACU | | | AAU | | | AGU | | | U |
| | | | | | | | | {close oversize bracket} | Asn | | {close oversize bracket} | Ser | |
| | AUC | {close oversize bracket} | Ile | ACC | | | AAC | | | AGC | | | C |
| | AUA | | | ACA | {close oversize bracket} | Thr | AAA | | | AGA | | | A |
| | | | | | | | | {close oversize bracket} | Lys | | {close oversize bracket} | Arg | |
| | AUG** | | Met | ACG | | | AAG | | | AGG | | | G |
| G | GUU | | | GCU | | | GAU | | | GGU | | | U |
| | | | | | | | | {close oversize bracket} | Asp | | | | |
| | GUC | | | GCC | | | GAC | | | GGC | | | C |
| | GUA | {close oversize bracket} | Val | GCA | {close oversize bracket} | Ala | GAA | | | GGA | {close oversize bracket} | Gly | A |
| | | | | | | | | {close oversize bracket} | Glu | | | | |
| | GUG** | | | GCG | | | GAG | | | GGG | | | G |

*Chain-terminating, or “nonsense” codon.
**Also used to specify the initiator formyl-Met-tRNAMet. The Val triplet GUG is therefore “ambiguous” in that it codes both valine and methionine.

The genetic code showing mRNA triplets and the amino acids for which they code.

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E. coli.

A second aspect of the invention provides an isolated polypeptide obtainable from a nucleic acid sequence according to the invention. As indicated above, the genetic code for translating a nucleic acid sequence into an amino acid sequence is well known.

Preferably the sequence comprises is a sequence encoding BUC11 (SEQ ID NO: 36), BUC6 (SEQ ID NO: 31), BUC9 (SEQ ID NO: 32) or BUC10 (SEQ ID NO: 35), preferably as defined above.

The invention further provides polypeptide analogues, fragments or derivatives of antigenic polypeptides which differ from naturally-occurring forms in terms of the identity of location of one or more amino acid residues (deletion analogues containing less than all of the residues specified for the protein, substitution analogues wherein one or more residues specified are replaced by other residues or addition analogues wherein one or more amino acid residues are added to a terminal or medial portion of the polypeptides) and which share some or all properties of the naturally-occurring forms. Preferably such polypeptides comprise between 1 and 20, preferably 1 and 10 amino acid deletions or substitutions.

Preferably the fragments comprise at least 4 amino acids of the sequence of BUC10 (SEQ ID NO: 36), BUC6 (SEQ ID NO: 31), BUC9 (SEQ ID NO: 32) or BUC10 (SEQ ID NO: 35). Preferably the fragment contains at least 10, at least 20, at least 30 contiguous amino acids, preferably all of the amino acids of BUC11 (SEQ ID NO: 36), BUC6 (SEQ ID NO: 31), BUC9 (SEQ ID NO: 32) or BUC10 (SEQ ID NO: 35).

Preferably the polypeptide is at least 80%, 85%, 90%, 95%, 96%, 97%, 98% or 99% identical to the sequences of the invention. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

Preferably the polypeptide is a fragment of BUC11 (SEQ ID NO: 36). This may have the sequence: PS KRLFFKKKRLC (SEQ ID NO: 1). This has been used to raise antibodies against BUC11 (SEQ ID NO: 36), in for example, rabbits.

The third aspect of the invention provides the use of nucleic acids or polypeptides according to the invention, to detect or monitor cancers, preferably breast cancer.

The expression of the isolated nucleic acid molecule has been found to be highly breast specific. This means that secondary tumours found elsewhere in the body may be characterised by expression of the molecule as being derived from what may be a previously undiagnosed primary breast tumour.

The use of a nucleic acid molecule hybridisable under high stringency conditions, to a nucleic acid according to the first aspect of the invention to detect or monitor cancers, e.g. breast cancer, melanoma, oesophageal cancer or gastric cancer, is also encompassed, as detailed above for the individual sequences. Such molecules may be used as probes, e.g. using PCR.

The expression of genes, and detection of their polypeptide products may be used to monitor disease progression during therapy or as a prognostic indicator of the initial disease status of the patient.

The presence of the nucleic acid molecule may also be used to characterise primary or secondary tumours as, for example, being derived from breast. This has implications for the assessment of the most suitable cancer treatment for the tumour as different types of tumour may be controlled by different drugs.

There are a number of techniques which may be used to detect the presence of a gene, including the use of Northern blot and reverse transcription polymerase chain reaction (RT-PCR) which may be used on tissue or whole blood samples to detect the presence of cancer associated genes. For polypeptide sequences in situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2nd Edition). ELISA based assays necessitate the use of antibodies raised against the protein or peptide sequence and may be used for the detection of antigen in tissue or serum samples (McIntyre C. A., Rees R. C. et. al.,

Europ. J. Cancer 28, 58-631 (1990)). In situ detection of antigen in tissue sections also rely on the use of antibodies, for example, immuno peroxidase staining or alkaline phosphatase staining (Gaepel, J. R., Rees, R. C. et. al. Brit. J. Cancer 64, 880-883 (1991)) to demonstrate expression. Similarly radio-immune assays may be developed whereby antibody conjugated to a radioactive isotope such as I¹²⁵ is used to detect antigen in the blood.

Blood or tissue samples may be assayed for elevated concentrations of the nucleic acid molecules or polypeptides.

Methods of producing antibodies which are specific to the polypeptides of the invention, for example, by the method of Kohler & Milstein to produce monoclonal antibodies, are well known. A further aspect of the invention provides an antibody, e.g. a monoclonal antibody, which specifically binds to a polypeptide according to the invention.

Kits for detecting or monitoring cancer, such as gastro-intestinal cancers, including breast cancer, using polypeptides, nucleic acids or antibodies according to the invention are also provided. Such kits may additionally contain instructions and reagents to carry out the detection or monitoring.

A fourth aspect of the invention provides for the use of nucleic acid molecules according to the first aspect of the invention or polypeptide molecules according to the second aspect of the invention in the prophylaxis or treatment of cancer, or pharmaceutically effective fragments thereof. By pharmaceutically effective fragment, the inventors mean a fragment of the molecule which still retains the ability to be a prophylactant or to treat cancer. The cancer may be a breast cancer.

The molecules are preferably administered in a pharmaceutically effective amount. Preferably the dose is between 1 mg/kg to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Polypeptides derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) causes the production of protein and subsequently activates the immune system. An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes or adaptive cellular therapy using, e.g., T-cells responsive to BUC peptides or BUC protein. A DNA based vaccine is demonstrated in, for example, Thompson S. A., et al. (J. Immunol. (1998), Vol. 160, pages 1717-1723).

Accordingly, the invention provides a nucleic acid molecule according to the invention in combination with a pharmaceutically-acceptable carrier.

Such polypeptides may be bound to a carrier molecule such as tetanus toxoid to make the polypeptide immunogenic. Such constructs are also within the scope of the invention.

A further aspect of the invention provides a method of prophylaxis or treatment of a cancer such as a breast cancer, comprising the administration to a patient of a nucleic acid molecule according to the invention.

The polypeptide molecules according to the invention may be used to produce vaccines to vaccinate against a cancer, such as a breast cancer.

Accordingly, the invention provides a polypeptide according to the invention in combination with a pharmaceutically acceptable carrier.

The invention further provides use of a polypeptide according to the invention in prophylaxis or treatment of a cancer such as a breast cancer.

Methods of prophylaxis or treating a cancer, such as a breast cancer, by administering a protein or peptide according to the invention to a patient, are also provided.

Vaccines comprising nucleic acid and/or polypeptides according to the invention are also provided. The polypeptide may be attached to another carrier peptide such as tetanus toxoid to increase the immogenicity of the polypeptide.

The polypeptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition phage display antibodies may be produced, this offers an alternative procedure to conventional hybridoma methodology. Having raised antibodies which may be of value in detecting tumour antigen in tissues or cells isolated from tissue or blood, their usefulness as therapeutic reagents could be assessed. Antibodies identified for their specific reactivity with tumour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy. Unconjugated antibodies can also be useful in influencing cancer cell growth. For example, the binding of certain antibodies to cell-surface receptors on cancer cells may initiate cell death by, e.g., apoptosis. Therefore the antibodies of this invention could be therapeutically useful in a non-conjugated form.

The term “antibody” includes intact antibody molecules as well as antibody fragments such as Fab, F(ab′)₂ and Fv.

The invention accordingly provides a method of treating a cancer such as breast cancer, by the use of one or more antibodies raised against a polypeptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labelled by techniques known in the art, e.g. with radioactive or fluorescent labels. Suitable nucleotide sequences for the probes include the sequences provided below:

| | |
|---|---|
| BUC 6 forward: | |
| 5′ TGGAAAATGTGTCCACCAAG | (SEQ ID NO: 2) |
| BUC 6 reverse: | |
| 5′ CGCTGCTGTAAGCATTCACT | (SEQ ID NO: 3) |
| BUC6(2) forward: | |
| 5′ ttgcataaagctgcacagga | (SEQ ID NO: 4) |
| BUC6(2) reverse: | |
| 5′ cttcaacacaacccccatgt | (SEQ ID NO: 5) |
| BUC6(3) forward: | |
| 5′ ccttcgagttcctttttctgg | (SEQ ID NO: 6) |
| BUC6(3) reverse: | |
| 5′ ggcaacacaaactcagagca | (SEQ ID NO: 7) |
| BUC9 forward: | |
| 5′ ccagattttcaccgctatgc | (SEQ ID NO: 8) |
| BUC9 reverse: | |
| 5′ aggcaagctctcatcaggac | (SEQ ID NO: 9) |
| BUC10 forward: | |
| 5′ caccgacgtttaaaggagga | (SEQ ID NO: 10) |
| BUC10 reverse: | |
| 5′ gtcctctgcaccttgggata | (SEQ ID NO: 11) |
| BUC11 forward: | |
| 5′ tctttcccacaatccctgac | (SEQ ID NO: 12) |
| BUC11 reverse: | |
| 5′ cagcttgccccatgtatttt | (SEQ ID NO: 13) |
| BUC11(2) forward: | |
| 5′ tgccattccactgttttctg | (SEQ ID NO: 14) |
| BUC11(2) reverse: | |
| 5′ cagcttgccccatgtatttt | (SEQ ID NO: 15) |
| BUC11(3) forward: | |
| 5′ tctagtcgacccacaatccctgacacagaa | (SEQ ID NO: 16) |
| BUC11(3) reverse: | |
| 5′ tctatctagagccagaaaacagtggaatgg | (SEQ ID NO: 17) |
| BUC6/9 forward: | |
| 5′ gaggccttgctaatttccta | (SEQ ID NO: 18) |
| BUC6/9 reverse: | |
| 5′ ctgttgcagtgagctcaagt | (SEQ ID NO: 19) |
| BUC9/10 forward: | |
| 5′ gtcctgatgagagcttgcct | (SEQ ID NO: 20) |
| BUC9/10 reverse: | |
| 5′ caaactggccttgatctgga | (SEQ ID NO: 21) |
| BUC6Q forward: | |
| 5′ ctcgaagccatcaatgacaa | (SEQ ID NO: 22) |
| BUC6Q reverse: | |
| 5′ tgagataatccgctccttgg | (SEQ ID NO: 23) |
| BUC9Q forward: | |
| 5′ gccacatggggtatgttctc | (SEQ ID NO: 24) |
| BUC9Q reverse: | |
| 5′ aggcaagctctcatcaggac | (SEQ ID NO: 25) |

Preferably the cancer which is detected, assayed for, monitored, treated or targeted for prophylaxis, is a breast cancer.

The invention will now be described by way of example only and with reference to the following example and figures.

FIG. 1: RT-PCR expression analysis of BUC6 (SEQ ID NO: 31) in normal tissues.

FIG. 2: RT-PCR expression analysis of BUC6 (SEQ ID NO: 31) in normal tissues.

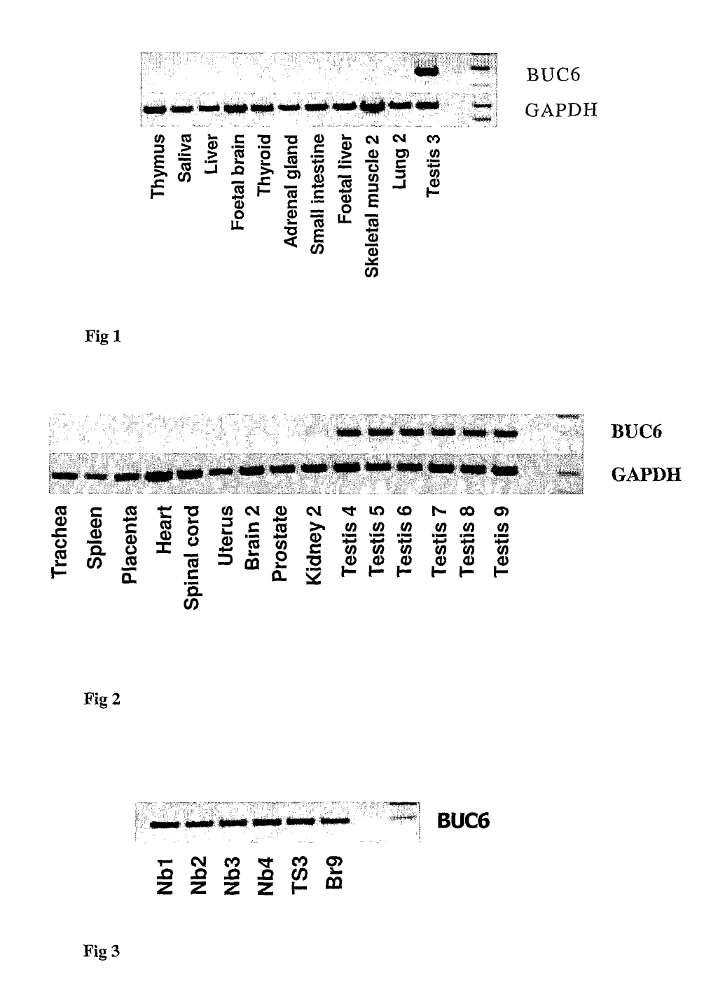

FIG. 3: RT-PCR expression analysis of BUC6 (SEQ ID NO: 31) in normal tissues and in breast cancer tissue. Nb: normal breast tissue; TS: normal testis tissue; Br: breast cancer tissue.

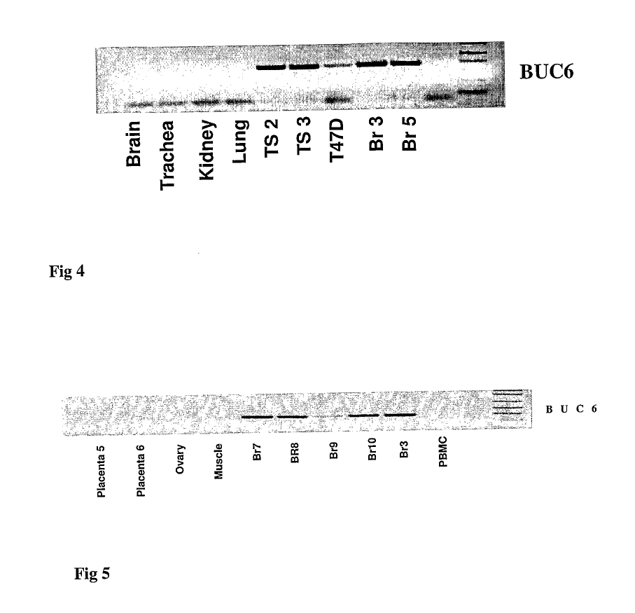

FIG. 4: Expression analysis of BUC6 (SEQ ID NO: 31) in normal tissues, testes, breast cancer tissues and breast cancer cell line by RT-PCR. TS: normal testis; Br: breast cancer tissue; T47D: breast cancer cell line.

FIG. 5: Expression analysis of BUC6 (SEQ ID NO: 31) in normal and breast cancer tissues by RT-PCR. PBMC: peripheral blood mononuclear cells; Br: breast cancer tissue.

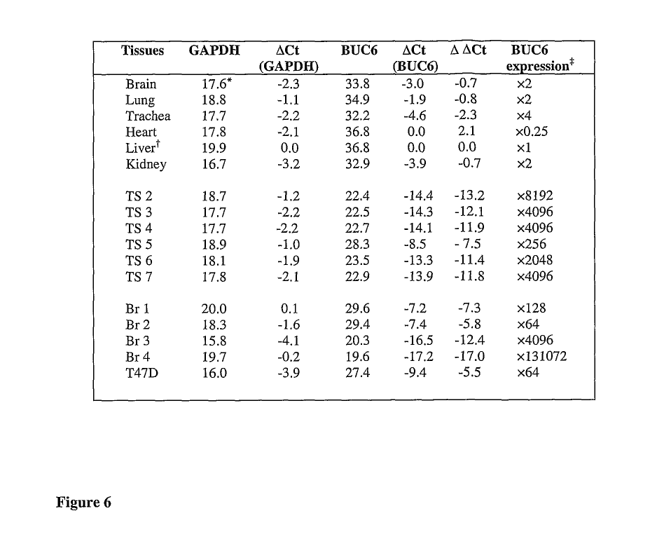

FIG. 6: Quantitative analysis of BUC6 (SEQ ID NO: 31) mRNA in normal and breast cancer tissues and breast cancer cell line. TS: normal testis; Br: breast cancer tissue; T47D: breast cancer cell line. *Ct value; †Reference tissue; ‡Compared with liver.

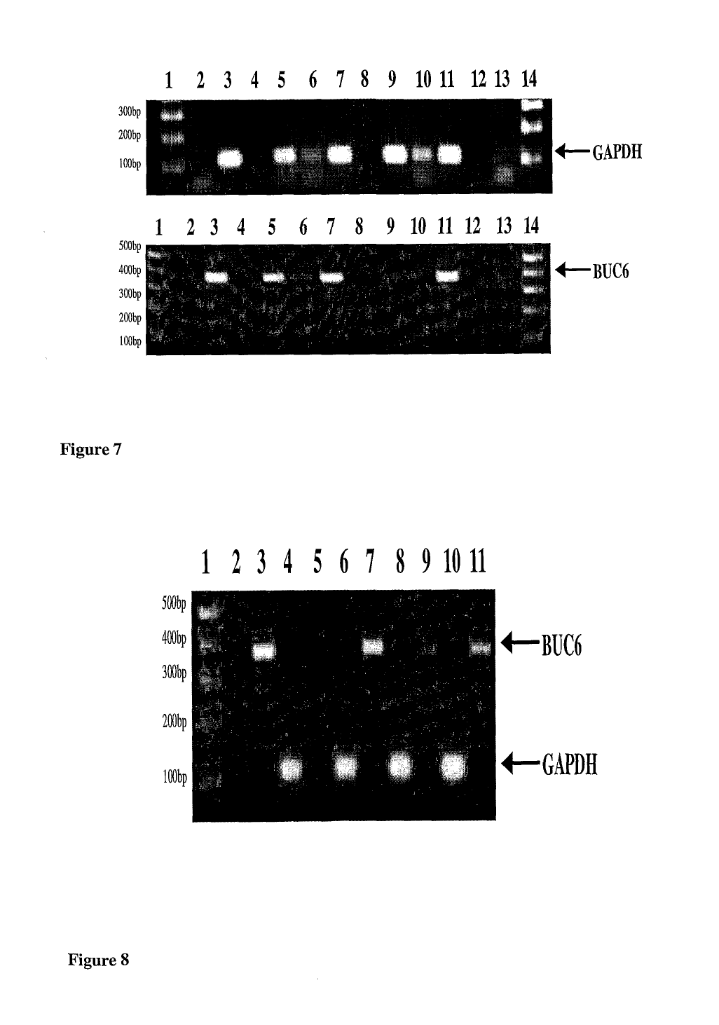

FIG. 7: mRNA expression of BUC6 (SEQ ID NO: 31) in melanoma samples. Lanes: (1) DNA ladder, (2) Negative control, (3) Positive control SK-BR3-V-, (4) to (13) Melanoma Ito 10, (14) DNA ladder. Following completion of the RT-PCR reaction, 24 μl of the products were loaded and run on a 1.5% Agarose gel. Bands were present at approximately 350 bp which correspond to the expected size (375 bp) of the target for the BUC6 (SEQ ID NO: 31) primers used.

FIG. 8: mRNA expression of BUC6 (SEQ ID NO: 31) in breast cancer cell lines. Lanes: (1) DNA ladder, (2) Negative control, (3) Positive control SK-BR3-V-, (4) and (5) BR293, (6) and (7) T47D, (8) and (9) MDA468, (10) and (11) MDA231.

Following completion of the RT-PCR reaction, 24 μl of the products were loaded and run on a 1.5% Agarose gel. Bands were present at approximately 350 bp which correspond to the expected size (375 bp) of the target for the BUC6 (SEQ ID NO: 31) primers used.

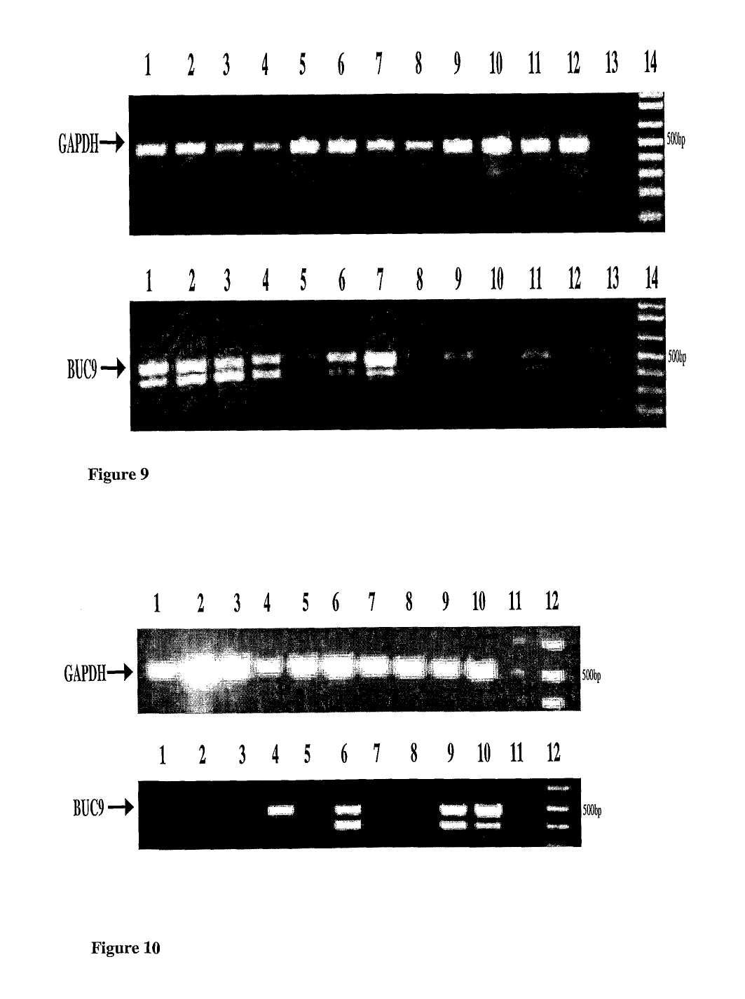

FIG. 9: mRNA expression of BUC9 (SEQ ID NO: 32) in breast cancer tissues and cell lines. Lanes: (1) to (8) Breast cancer tissues: (1) BR11, (2) BR12, (3) BR13, (4) BR15, (5) BR16, (6) BR19, (7) BR25, (8) BR28. (9) to (12): breast cancer cell lines: (9) MDAP3, (10) MDA435, (11) T47D, (12) BR293. (13) Negative control, (14) DNA ladder.

Following completion of the RT-PCR reaction, products were loaded and run on a 1.5% Agarose gel. Bands were present at approximately 500 bp which correspond to the expected size (495 bp) of the target for the BUC9 (SEQ ID NO: 32) primers used. The double band observed suggests that BUC9 (SEQ ID NO: 32) may exist as a splice variant in breast cancer tissues and cell lines except for MDAP3.

FIG. 10: mRNA expression of BUC9 (SEQ ID NO: 32) in normal tissues. Lanes: (1) Brain, (2) Skeletal muscle, (3) Kidney, (4) Placenta, (5) Heart, (6) Testis, (7) Lung, (8) Liver, (9) Breast 1, (10) Breast 2, (11) Negative control, (12) DNA ladder.

Following completion of the RT-PCR reaction, products were loaded and run on a 1.5% Agarose gel. Bands were present at approximately 500 bp which correspond to the expected size (495 bp) of the target for the BUC9 (SEQ ID NO: 32) primers used. The double band observed for testis and the breast samples suggest that BUC9 (SEQ ID NO: 32) may exist as a splice variant in these tissues.

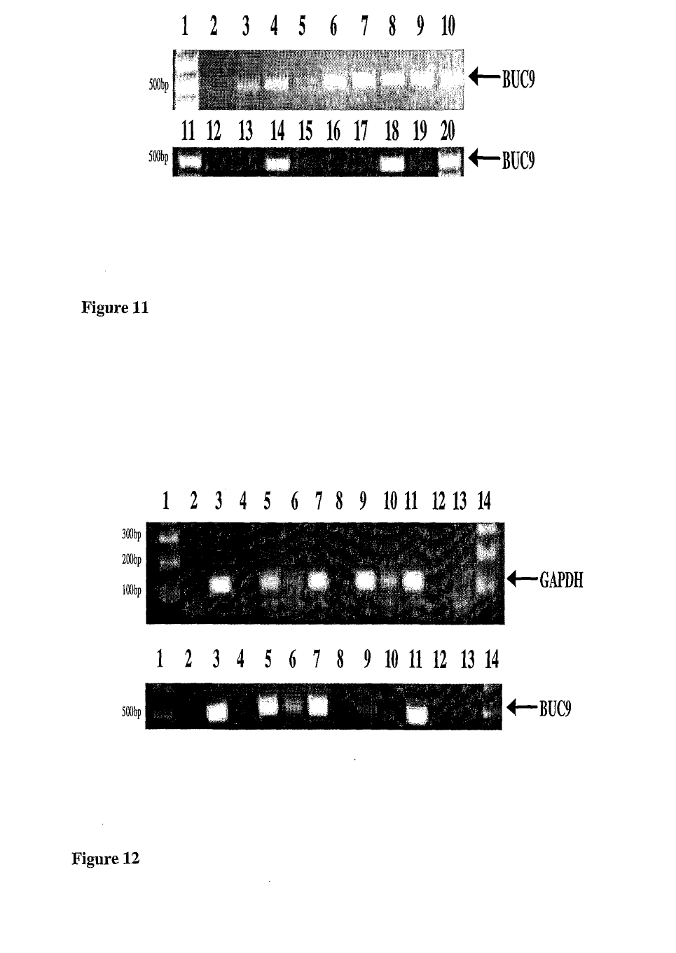

FIG. 11: mRNA expression of BUC9 (SEQ ID NO: 32) in paired oesophageal normal and cancer tissues and paired gastric normal and cancer tissues. Lanes: (1) DNA ladder, (2) Negative control, (3) Normal oesophageal 2, (4) Oesophageal cancer 2, (5) Normal oesophageal 1, (6) Oesophageal cancer 1, (7) Normal oesophageal 3, (8) Oesophageal cancer 3, (9) Normal oesophageal 4, (10) Oesophageal cancer 4, (11) DNA ladder, (12) Normal gastric 2, (13) Gastric cancer 2, (14) Normal gastric 4, (15) Gastric cancer 4, (16) Normal gastric 5, (17) Gastric cancer 5, (18) Normal gastric 6, (19) Gastric cancer 6, (20) DNA ladder.

Following completion of the RT-PCR reaction, products were loaded and run on a 1.5% Agarose gel. Bands were present at approximately 500 bp which correspond to the expected size (495 bp) of the target for the BUC9 (SEQ ID NO: 32) primers used.

FIG. 12: mRNA expression of BUC9 (SEQ ID NO: 32) in melanoma samples. Lanes: (1) DNA ladder, (2) Negative control, (3) Positive control SK-BR3-V-, (4) to (13) Melanoma 1 to 10, (14) DNA ladder.

Following completion of the RT-PCR reaction, 24 μl of the products were loaded and run on a 1.5% Agarose gel. Bands were present at approximately 500 bp which correspond to the expected size (495 bp) of the target for the BUC9 (SEQ ID NO: 32) primers used.

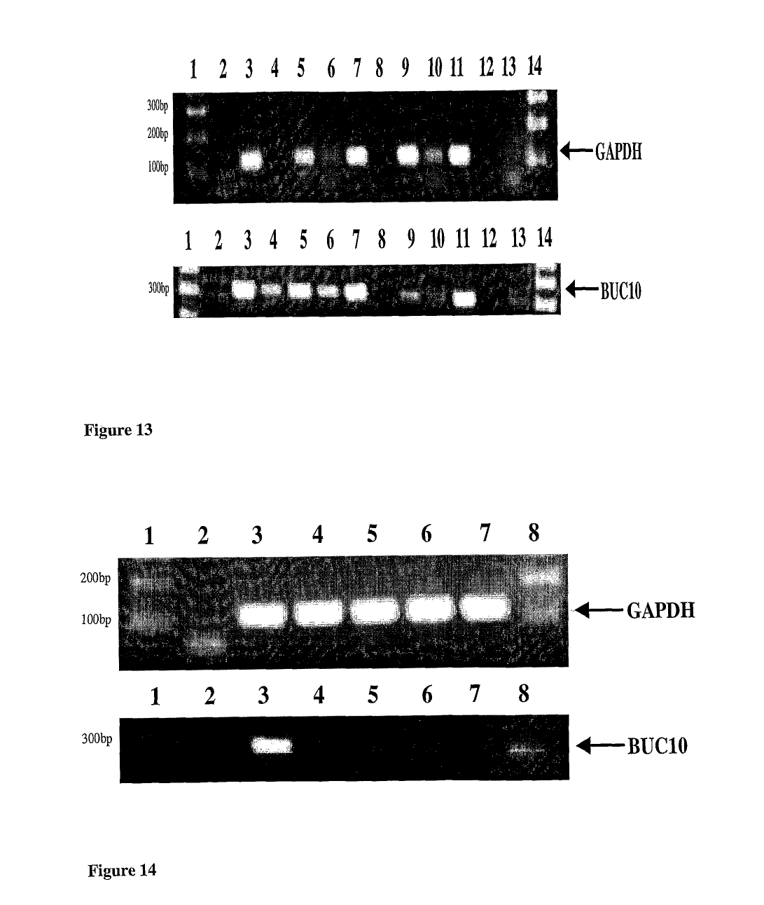

FIG. 13: mRNA expression of BUC10 (SEQ ID NO: 35) in melanoma samples. Lanes: (1) DNA ladder, (2) Negative control, (3) Positive control SK-BR3-V-, (4) to (13) Melanoma 1 to 10, (14) DNA ladder.

Following completion of the RT-PCR reaction, 24 μl of the products were loaded and run on a 1.5% Agarose gel. Bands were present at approximately 300 bp which correspond to the expected size (304 bp) of the target for the BUC10 (SEQ ID NO: 35) primers used.

FIG. 14: mRNA expression of BUC10 (SEQ ID NO: 35) in breast cancer cell lines. Lanes: (1) DNA ladder, (2) Negative control, (3) Positive control SK-BR3-V-, (4) BR293, (5) T47D, (6) MDA468, (7) MDA231, (8) DNA ladder.

Following completion of the RT-PCR reaction, products were loaded and run on a 1.5% Agarose gel. Bands were present at approximately 300 bp which correspond to the expected size (304 bp) of the target for the BUC10 (SEQ ID NO: 35) primers used.

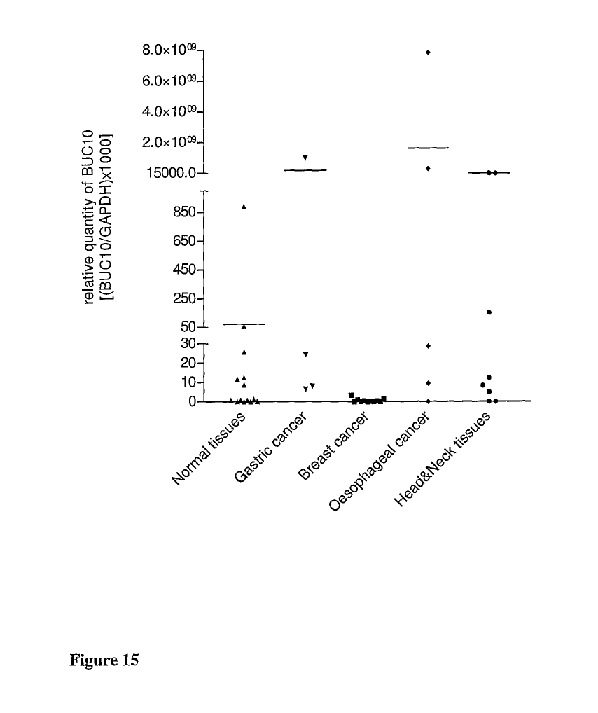

FIG. 15: A scatter graph of mRNA BUC10 (SEQ ID NO: 35) gene expression in normal tissues, gastric cancer tissues, breast cancer tissues, oesophageal cancer tissues and head and neck (cancer and normal) tissues using RT-Q-PCR. The SQ mean of BUC10 (SEQ ID NO: 35) was divided by the SQ mean of GAPDH and the result was multiply by 1000.

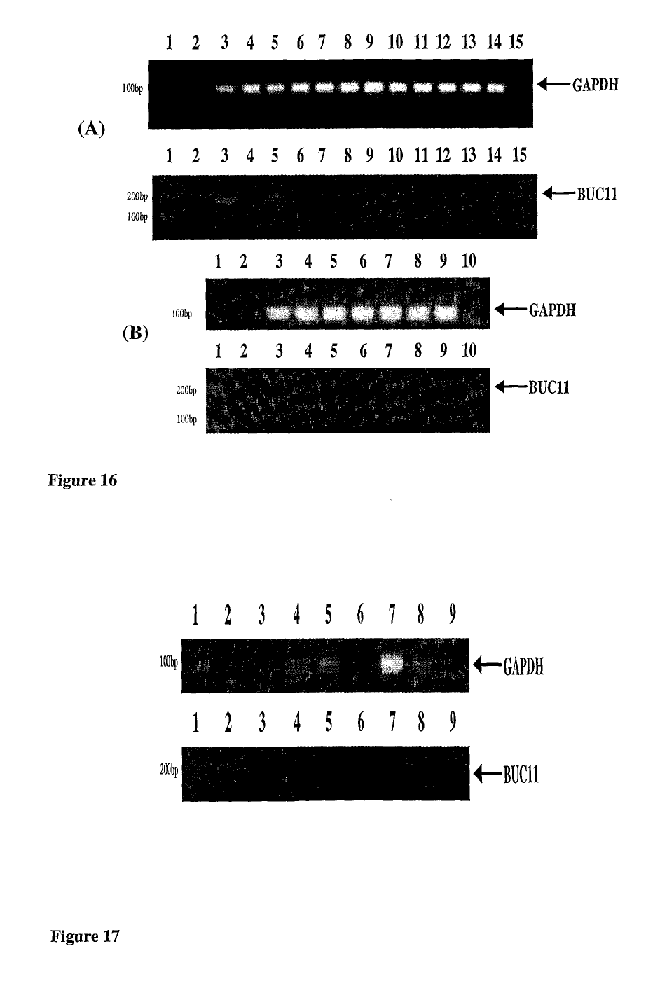

FIG. 16: mRNA expression of BUC11 (SEQ ID NO: 36) in normal tissues. Figure (A) lanes: (1) DNA ladder, (2) Negative control, (3) Breast, (4) Heart, (5) Testis, (6) Liver, (7) Prostate, (8) Brain, (9) Uterus, (10) Spleen, (11) Skeletal muscle, (12) Lung, (13) Kidney, (14) Placenta, (15) DNA ladder. Figure (B) lanes: (1) DNA ladder, (2) Negative control, (3) Fetal brain, (4) Fetal liver, (5) Trachea, (6) Thyroid, (7) Spinal cord, (8) Salivary gland, (9) Thymus, (10) DNA ladder.

Following completion of the RT-PCR reaction, products were loaded and run on a 1.5% Agarose gel. Bands were present at approximately 200 bp which correspond to the expected size (197 bp) of the target for the BUC11 (SEQ ID NO: 36) primers used.

FIG. 17: mRNA expression of BUC11 (SEQ ID NO: 36) in Peripheral Blood Mononuclear Cells (PBMC). Lanes: (1) DNA ladder, (2) Negative control, (3) to (8) PBMC: (3) PBMC1, (4) PBMC2, (5) PBMC3, (6) PBMC4, (7) PBMC83, (8) PBMC108, (9) DNA ladder.

Following completion of the RT-PCR reaction, products were loaded and run on a 1.5% Agarose gel. BUC11 (SEQ ID NO: 36) mRNA is not expressed in any of the PBMC tested. The band which appears in lane 2 for BUC11 (SEQ ID NO: 36) is the DNA ladder that has been accidentally loaded twice.

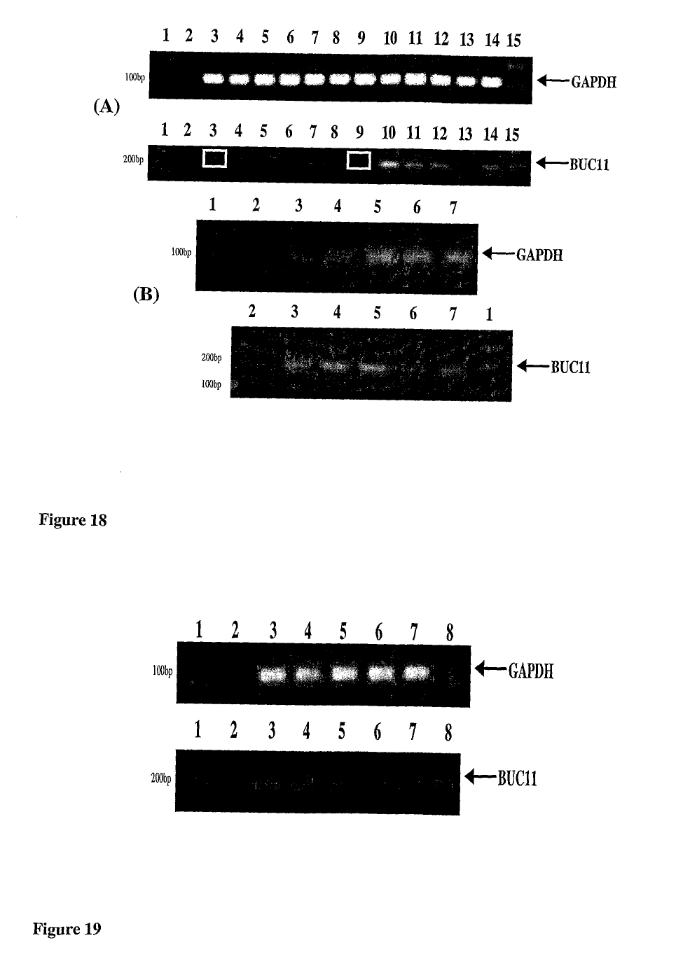

FIG. 18: mRNA expression of BUC11 (SEQ ID NO: 36) in breast cancer cell lines and tissues. Figure (A) lanes: (1) DNA ladder, (2) Negative control, (3) to (8) cell lines: (3) T47D, (4) MDA231, (5) MDA468, (6) BR293, (7) MCF-7, (8) MDAP3, (9) to (14) tissues: (9) BR2, (10) BR15, (11) BR19, (12) BR11, (13) BR20, (14) BR13, (15) DNA ladder. Figure (B) lanes: (1) DNA ladder, (2) Negative control, (3) to (7) tissues: (3) BR25, (4) BR26, (5) BR27, (6) BR9, (7) BR12.

Following completion of the RT-PCR reaction, products were loaded and run on a 1.5% Agarose gel. Bands were present at approximately 200 bp which correspond to the expected size (197 bp) of the target for the BUC11 (SEQ ID NO: 36) primers used.

FIG. 19: mRNA expression of BUC11 (SEQ ID NO: 36) in testis (normal and cancer) tissues. Lanes: (1) DNA ladder, (2) Negative control, (3) to (5) testicular cancer tissues: (3) TS2, (4) TS3, (5) TS4, (6) to (7) normal testis tissues: (6) TS6, (7) TS7, (8 DNA ladder.

Following completion of the RT-PCR reaction, products were loaded and run on a 1.5% Agarose gel. Bands were present at approximately 200 bp which correspond to the expected size (197 bp) of the target for the BUC11 (SEQ ID NO: 36) primers used.

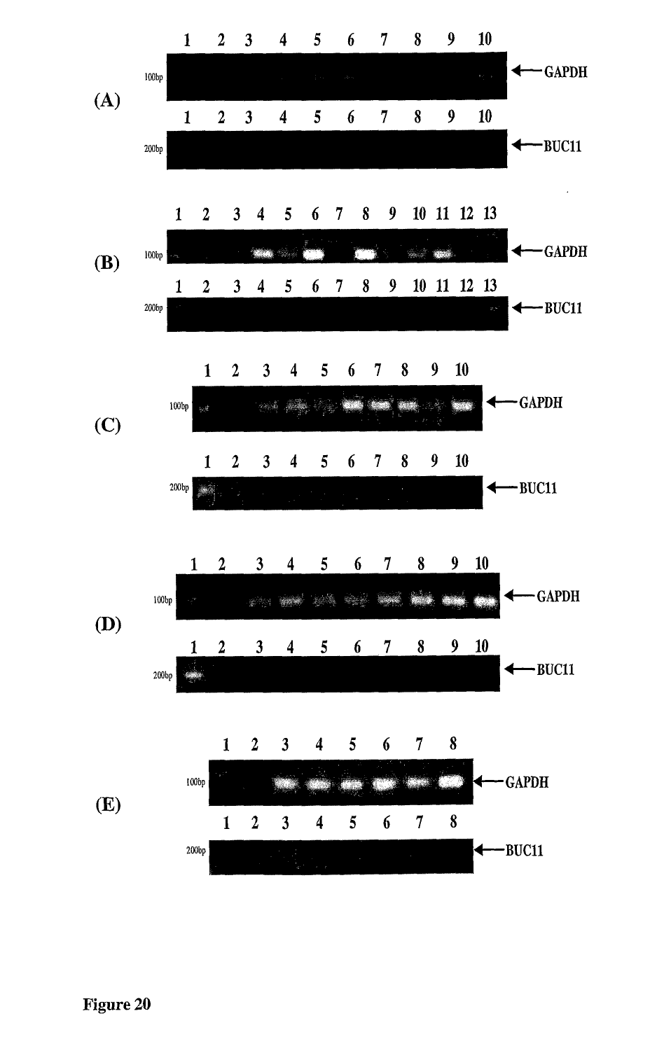

FIG. 20: mRNA expression of BUC11 (SEQ ID NO: 36) in diverse cancer tissues. Figure (A) lanes: (1) DNA ladder, (2) Negative control, (3) to (10) Mesothelioma tissues: (3) Meso1, (4) Meso2, (5) Meso3, (6) Meso4, (7) Meso5, (8) Meso6, (9) Meso7, (10) Meso8.

Figure (B) lanes: (1) DNA ladder, (2) Negative control, (3) to (12) Melanoma samples 1 to 10, (13) DNA ladder.

Figure (C) lanes: (1) DNA ladder, (2) Negative control, (3) to (10) Gastric paired normal (N) and cancer (T) tissues: (3) BN2, (4) BT2, (5) BN4, (6) BT4, (7) BN5, (8) BT5, (9) BN6, (10) BT6.

Figure (D) lanes: (1) DNA ladder, (2) Negative control, (3) to (10) Kidney paired normal (N) and cancer (T) tissues: (3) KN1, (4) KT1, (5) KN2, (6) KT2, (7) KN10, (8) KT10, (9) KN12, (10) KT12.

Figure (E) lanes: (1) DNA ladder, (2) Negative control, (3) to (8) Oesophageal paired normal (N) and cancer (T) tissues: (3) EN1, (4) ET1, (5) EN3, (6) ET3, (7) EN4, (8) ET4.

Following completion of the RT-PCR reaction, products were loaded and run on a 1.5% Agarose gel. BUC11 (SEQ ID NO: 36) mRNA is not expressed in any of the tissues tested. This experiment is semi-quantitative for (A), (C), (D) and (E) but not for (B).

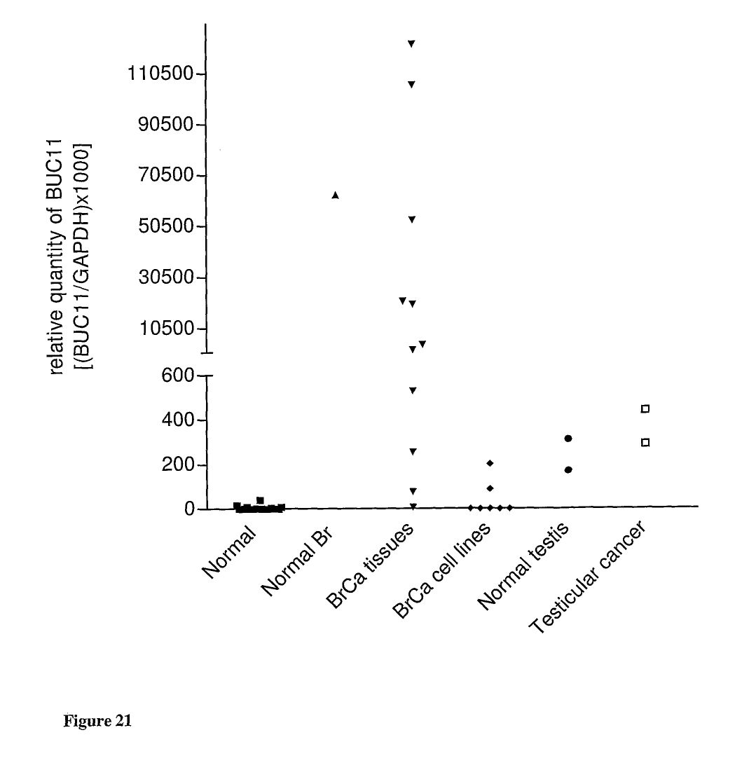

FIG. 21: A scatter graph of mRNA BUC11 (SEQ ID NO: 36) gene expression in normal tissues, breast cancer cell lines and tissues, and testicular cancer using RT-Q-PCR. The SQ mean of BUC11 (SEQ ID NO: 36) was divided by the SQ mean of GAPDH and the result was multiply by 1000.

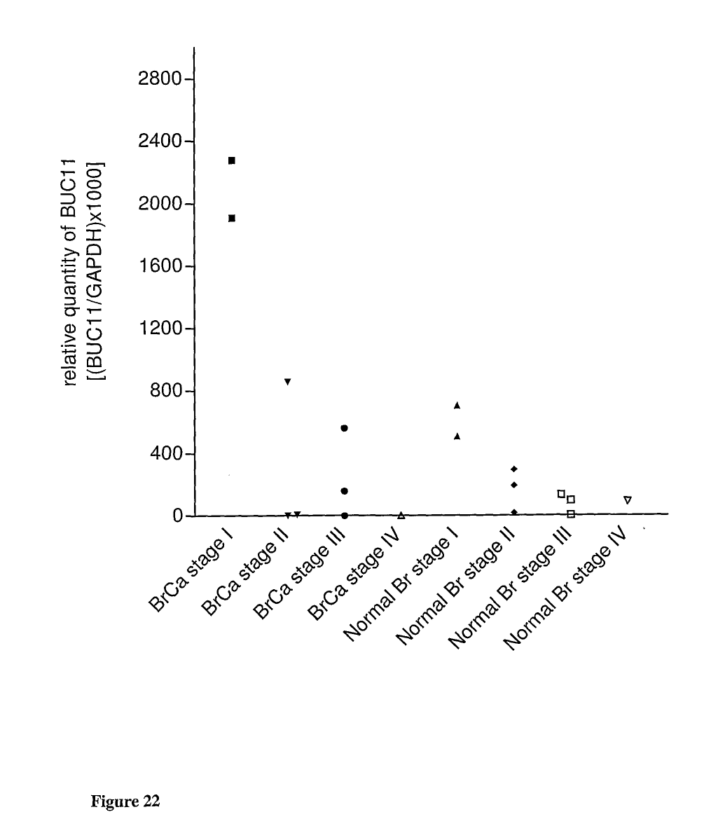

FIG. 22: A scatter graph of mRNA BUC11 (SEQ ID NO: 36) gene expression in breast cancer tissues from different stages using RT-Q-PCR. RNA samples have been provided by Dr. Aija Line the Latvian University Biomedical Centre (Latvia). The RNA was extracted from paraffin-embedded tissues and reverse-transcribed using Random Primers. The tissues are paired cancer and normal counterpart, each pair from the same patient. The SQ mean of B

FIG. 23: mRNA sequence of BUC6 (SEQ ID NO: 31).

FIG. 24(a): mRNA sequence of BUC9 (SEQ ID NO: 32).

FIG. 24(b): Two potential variants of BUC9 (SEQ ID NO: 32).

Following RT-PCR with BR15 cDNA, the gel extracted and purified “upper band” (about 500 bp) and “lower band” (about 400 bp) were individually cloned into pCR11-blunt-TOPO vectors and XL1B cells were transformed and cultured. After DNA Miniprep and EcoRI restriction digest, positive clones for BUC9 (SEQ ID NO: 32) genes were sent to sequencing. The sequences were then aligned to the original BUC9 (SEQ ID NO: 32) sequence using the JustBio Aligner tool (http://www.justbio.com). 8H1M13Forward (SEQ ID NO: 33) shows the “upper band” sequence and 4LM13Forward (SEQ ID NO: 34) shows the “lower band” sequence. The 4LM13Forward (SEQ ID NO: 34) sequence clearly lack of about 100 bp which are present in 8H1M13Forward (SEQ ID NO: 33) sequence. These sequences represent two potential splice variants for BUC9 (SEQ ID NO: 32).

FIG. 25: mRNA sequence of BUC10 (SEQ ID NO: 35).

FIG. 26: mRNA sequence of BUC11 (SEQ ID NO: 36).

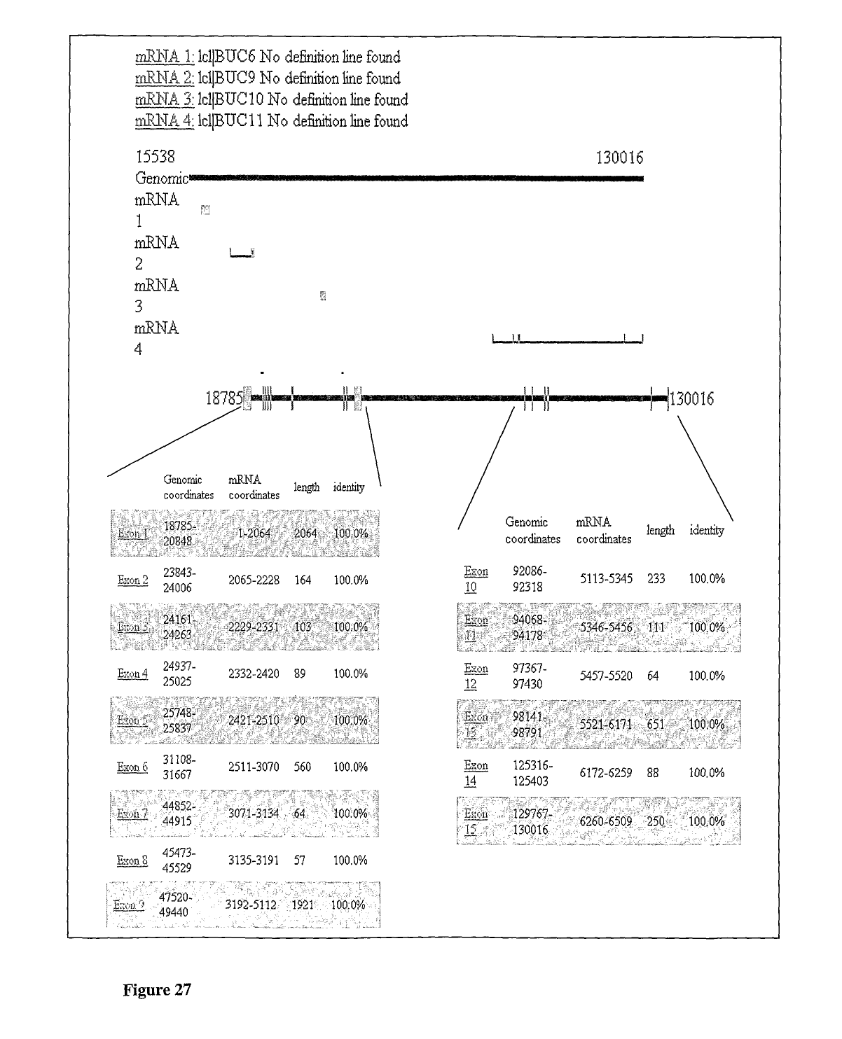

FIG. 27: SPIDEY alignments of BUC mRNA original sequences and sequencing results against genomic Human DNA sequence from clone RP11-20F24 on chromosome 10p11.21-12.1. These alignments were made in SPIDEY alignment tool (http://www.ncbi.nlm.nih.gov/spidey/). According to a database research, the BUC genes may all be related (top alignment). Genomic DNA corresponds to Human DNA sequence from clone RP11-20F24 on chromosome 10p11.21-12.1 which contains the 3′ end of the NY-BR-1 gene for breast cancer antigen NY-BR-1, two novel genes, a pseudogene similar to part of ATP8. RT-PCR has been carried out using BUC6 (SEQ ID NO: 31), BUC9 (SEQ ID NO: 32), BUC10 (SEQ ID NO: 35), BUC11 (SEQ ID NO: 36) primers. PCR products were sequenced and sequences aligned. Overlapping of the sequences was observed. The “BUC genes” are expressed as an mRNA sequence of 6509 base-pair long and which does not include any obvious protein coding frame.

FIG. 28: Alignment of the nucleotide sequences for BUC6 (SEQ ID NO: 31), 9 (SEQ ID NO: 32), 10 (SEQ ID NO: 35) and 11 (SEQ ID NO: 36).

FIG. 29: Amino acid sequence of the aligned BUC genes. Sequence of the predicted protein of the LOC646360 gene: mgllpppskr Iffkkkrlcf dlkyfiypqs qdtvdgikak hickkdiagg gakswkpslq swekqwygka meraf (SEQ ID NO: 26).

FIG. 30: BUC11 (SEQ ID NO: 36) mRNA sequence including the mRNA sequence of LOC646360. After a BLAST search (http://www.ncbi.nlm.nih.gov/BLAST/) and alignment of the sequences, the mRNA sequence of an unpublished hypothetical protein coding gene LOC646360 (in bold) is 100% matching the mRNA sequence of the last 6 exons of the “BUC gene” which includes BUC11 (SEQ ID NO: 36). The LOC646360 gene is located just after the NY-BR-1 gene, previously published regarding breast cancer, on chromosome 10 (10p11.21). LOC646360 has a predicted protein of 75 amino acids (aa). LOC646360 gene does not have an equivalent in any other species

### METHODOLOGY

### Bioinformatic Identification of Breast-Associated UniGene Clusters

The UniGene resource was used to search the UniGene clusters associated with human breast in the following manner. Firstly, UniGene clusters containing ESTs from cDNA libraries constructed from human breast were identified. Secondly, any of the selected UniGene clusters containing additional ESTs derived from essential human tissues or organs, such as brain, lung, liver, kidney, heart and pancreas except testis, ovary and placenta were excluded from further study. Thirdly, the UniGene clusters containing ESTs exclusively from normal human breast, and/or breast cancer tissues, and/or breast cancer cell lines were chosen.

### RT-PCR Analysis

Total RNA from different normal human tissues was purchased from Clontech (Oxford, UK). These included brain, trachea, kidney, lung, placenta, ovary, muscle, spleen, heart, spinal chord, uterus, prostate, thymus, saliva, liver, foetal, brain, thyroid, adrenal gland and small intestine. Normal breast and breast tumour tissues were derived from surgical specimens obtained from Saint Savvas Hospital (Athens, Greece). The Ethical Committee of the hospital approved the specimen collection procedure. Total RNA from the tissues was prepared by the guanidinium thiocyanate method (Sambrook, Molecular Cloning, a laboratory manual, 2^{nd} Ed.). The first-strand cDNA was synthesised from 2 μg of total RNA primed with oligo-dT¹⁵ primers (Promega, Southampton, UK). Gene specific PCR primers located within different exons were designed to amplify cDNA fragments of the selected UniGene clusters and from Glyceraldehyde-3-phosphate dehydrogenase (GAPDH, used as an internal control) in a total reaction volume of 50 μl. The reaction mixture contained 400 nM of each primer, 200 μM of dNTPs and 2.5 U of Taq polymerase in a thermal cycler (Perkin-Elmer, Beaconsfield, UK). The PCR products were analysed by agarose gel electrophoresis and stained with ethidium bromide. Amplification of BUC6 (SEQ ID NO: 31) was performed using the cycling conditions (45 seconds at 94° C., 45 seconds at 58° C., 45 seconds at 72° C.). The cycling conditions for all other BUC genes are as follows (30 seconds at 94° C., 30 seconds at 58° C., 30 seconds at 72° C.) except for the number of cycles. The appropriate number of cycles was determined for each target gene. The primers of BUC genes and GAPDH were as follows:

| | |
|---|---|
| GAPDH forward: | |
| 5′ GAGTCAAC-GGATTTGGTCGT | (SEQ ID NO: 27) |
| GAPDH reverse: | |
| 5′ AATGAAGGGGTCATTGATGG | (SEQ ID NO: 28) |

### Real-Time Quantitative RT-PCR

Total RNA and reverse transcription were prepared and carried out as described above. Gene-specific primers for real time PCR were designed using Primer 3 software (Primer 3 software is available at http://frodo.wi.mit.edu/cgi-bin/primer3/primer3_www.cgi) and synthesised commercially (Sigma-Genosys, Cambridge, UK).

iQ SYBR® green SuperMix (containing SYBR® green I dye, hot-start iTaq DNA polymerase, optimised buffer, and dNTPs) was purchased from Bio-Rad (Hemel Hempstead, UK). A 25 μl PCR reaction was prepared for each cDNA sample. Each PCR reaction consisted of 40 cycles of 95° C. for 15 seconds, 58° C. for 20 seconds and 72° C. for 30 seconds. The thermal cycling and fluorescent monitoring were performed using an iCycler iQ real-time PCR detection system (Bio-Rad). The cycle interval at which a PCR product is first detected above a fixed threshold, termed the cycle threshold (Ct), was determined for each sample. The Quantitative PCR (Q-PCR) results were analysed by the 2-ΔΔCT method 5). GAPDH was used as an internal control and normal human liver was used as a reference tissue. The primers used for real-time RT-PCR were the same as those used for the conventional RT-PCR, described above.

Molecular Cloning of BUC6 (SEQ ID NO: 31) Gene by Screening a Breast Cancer cDNA Library with Gene-Specific Probe.

In order to construct a breast cancer cDNA library, poly (A)+ RNA was isolated and purified from a breast cancer tissue. cDNA prepared from poly (A)+ RNA was ligated into the ZAP Expression vector using Gigapack III Gold cloning kit (Stratagene, Amsterdam, The Netherlands). After in vitro packaging, a cDNA library containing 2.0×106 primary cDNA clones was obtained.

Approximately 5000 phage plaques were screened on a nylon membrane using a digoxigenein (DIG) labelled PCR product of BUC6 (SEQ ID NO: 31). Those recombinant phage which were identified as positive recombinant phages were excised into phagemid (12). cDNA insert sequencing was performed in both directions using BigDye Terminator Cycle Sequencing Ready Reaction kit on ABI PRISM 310 automatic sequencer (Applied Biosystems, Foster City, Calif., USA).

### Results

### Identification of Breast-Associated UniGene Clusters by Database Mining

There were 18545 UniGene clusters containing EST(s) derived from cDNA libraries constructed from normal breast or breast cancer tissues. Among these were 228 UniGene clusters comprising ESTs from normal breast and/or breast cancer tissues or cell lines but not from normal essential tissues or organs except for testis, ovary and placenta. These 228 UniGene clusters have been designated as breast-associated UniGene clusters (BUC). The inventors have designated these clusters as BUC 1-228.

Expression Analysis of Breast-Associated UniGene Clusters by Conventional RT-PCR

The mRNA expression patterns of 4 selected UniGene clusters (BUC6 (SEQ ID NO: 31), BUC9 (SEQ ID NO: 32), BUC10 (SEQ ID NO: 35), BUC11 (SEQ ID NO: 36)) were analysed by conventional RT-PCR using a panel of RNA samples derived from normal tissues, breast cancer tissues, breast cancer cell lines, melanoma tissues, oesophageal cancer tissues, gastric cancer tissues, head & neck cancer tissues, peripheral blood mononuclear cells (PBMC), testis cancer tissues, kidney cancer tissues and mesothelioma tissues.

The mRNA expression patterns of the first five UniGene clusters (which the inventors annotated as BUC1-BUC5) revealed that they are ubiquitously expressed in normal tissues or could not be amplified (data not shown). Therefore, BUC1-BUC5 were excluded from further study. BUC6 (SEQ ID NO: 31) was found to be related to the cluster in UniGene accession number: hs.373787. This contains 56 ESTs, 17 of them are derived from breast tumour cDNA libraries, 25 from normal breast cDNA libraries, 4 from a normal testis library, 4 from a cDNA library constructed from pooled human tissues including testis, 1 from a cDNA library constructed from mixed foetal liver and spleen and 3 from vascular tissues (these 3 ESTs have the same location but have different sizes). The inventors identified that BUC 6 (SEQ ID NO: 31) maps to chromosome 10p11.21 in humans. The inventors found that some ESTs of BUC6 (SEQ ID NO: 31) are identical to human breast cancer antigen NY-BR-1 mRNA (6) and ankyrin repeat domain 30A (ANKRD30A) mRNA. However, the inventors found that most BUC6 (SEQ ID NO: 31) EST's are not homologous to any previously known genes.

PCR primers were designed to amplify the unique region of BUC6 (SEQ ID NO: 31) (i.e. BUC6 forward and BUC6 reverse sequences given below). As shown in FIGS. 1-6, BUC6 (SEQ ID NO: 31) was not expressed in 21 different normal foetal and adult tissues including brain, liver, lung, kidney and PBMC, but BUC6 (SEQ ID NO: 31) was highly expressed in normal breast and testis tissues, and breast cancer tissues. BUC6 (SEQ ID NO: 31) was also expressed in a breast cancer cell line (T47D). As shown in FIGS. 7-8, BUC6 (SEQ ID NO: 31) was expressed in all the melanoma samples tested and was also expressed in all the breast cancer cell lines tested.

PCR primers were designed to amplify the unique region of BUC9 (SEQ ID NO: 32) (i.e. BUC9 forward and BUC9 reverse, sequences given below). A shown in FIGS. 9-12, BUC9 (SEQ ID NO: 32) was expressed in 6 out of 9 breast cancer tissues tested, in 2 out of 4 breast cancer cell lines tested, in placenta, in normal testis, in normal breast, in all the tested oesophageal (paired normal and cancer) tissues, in 2 out of 4 gastric (paired normal and cancer) tissues and in all the melanoma samples tested.

PCR primers were designed to amplify the unique region of BUC10 (SEQ ID NO: 35) (i.e. BUC10 forward and BUC10 reverse, sequences given below). As shown in FIGS. 13-14, BUC10 (SEQ ID NO: 35) was expressed in all the melanoma samples tested and in all breast cancer cell lines tested but BR293.

PCR primers were designed to amplify the unique region of BUC11 (SEQ ID NO: 36) (i.e. BUC11 forward and BUC11 reverse, sequences given below). As shown in FIGS. 16-20, BUC11 (SEQ ID NO: 36) was expressed in normal breast tissue, normal testis tissue, in the breast cancer cell line T47D, in 90% of the breast cancer tissues tested, in all testis (paired normal and cancer) tissues tested, and BUC11 (SEQ ID NO: 36) was not expressed in any of the PBMC tested nor in any of the mesothelioma tissues, melanoma tissues, gastric (paired normal and cancer) tissues and oesophageal (paired normal and cancer) tissues tested.

Sequences for BUC 6 (SEQ ID NO: 31), 9, 10 and 11 are shown in FIGS. 23-26.

### Quantitative Analysis of BUC6 (SEQ ID NO: 31), BUC10 (SEQ ID NO: 35) and BUC11 (SEQ ID NO: 36) Gene Expression

To investigate further the mRNA expression profiles of BUC6 (SEQ ID NO: 31), quantitative real-time RT-PCR was performed using an RNA panel derived from various normal and tumour specimens. The normalised level of BUC6 (SEQ ID NO: 31) mRNA expression in normal and breast cancer tissues, relative to its expression level in normal liver, is given in FIG. 6. Overall, real-time RT-PCR analysis revealed very low levels of BUC6 (SEQ ID NO: 31) mRNA in normal tissues except testes. This is shown in FIGS. 1 and 2. The expression of BUC6 (SEQ ID NO: 31) in all breast cancer tissues tested was up-regulated. The level of BUC6 (SEQ ID NO: 31) in one breast cancer tissue (Br 4) was 131 072 times the level detected in normal liver.

A peptide sequence encoding a fragment of BUC11 (SEQ ID NO: 36) has been used to raise anti-BUC11 antibodies in rabbits:

| | |
|---|---|
| PS KRLFFKKKRLC | (SEQ ID NO: 1) |

BUC6 (SEQ ID NO: 31) was found to be breast specific. The relatively low levels observed in breast cancer cell lines (FIGS. 4 and 5) were due to tumour heterogeneity.

As shown in FIG. 15, BUC10 (SEQ ID NO: 35) mRNA was not overexpressed in breast cancer, when compared to its expression in the other cancer and normal tissues. BUC10 (SEQ ID NO: 35) mRNA was expressed at different levels in normal tissues, gastric cancer tissues, breast cancer tissues and oesophageal cancer tissues.

As shown in FIG. 21, BUC11 (SEQ ID NO: 36) mRNA was expressed at widely different levels in the breast cancer samples tested. BUC11 (SEQ ID NO: 36) mRNA was not (or at a very low level) expressed in the normal tissues tested except breast and testis tissues. BUC10 (SEQ ID NO: 36) mRNA was expressed at similar levels in the normal testis and testicular cancer tissues tested. BUC11 (SEQ ID NO: 36) mRNA was only expressed in the breast cancer cell lines T47D and MDA231.

As shown on FIG. 22, BUC11 (SEQ ID NO: 36) mRNA expression was higher in early stages of breast cancer when compared to later stages. In the normal breast tissues coming from the same patients, BUC11 (SEQ ID NO: 36) mRNA expression followed the same trend.

### BUC11 (SEQ ID NO: 36) Proliferation Assay

siRNA was designed for specific BUC11 (SEQ ID NO: 36) silencing. BUC11 (SEQ ID NO: 36) siRNA efficacy was firstly tested using Real-Time RT-PCR following transfection and mRNA isolation. The transfection of breast cancer cell line MDA231 was carried out using INTERFERin siRNA Transfection reagent (Autogen Bioclear, UK). The experiment was performed in duplicate wells. Each experiment comprised cells with BUC11 (SEQ ID NO: 36) gene-specific siRNA, cells with negative control siRNA, cells with INTERFERin alone and cells alone.

On day 2, 3H-thymidine (Sigma-Aldrich, UK) was added to the cells. On day 3, cell suspensions were transferred to a filter plate, Microscint solution (Packard, USA) was added and the reading of the plate was performed. The procedure was repeated on day 7 and day 10.

siRNA silencing of BUC11 (SEQ ID NO: 36) was observed to inhibit the proliferation of MDA231 breast cancer cells. This suggests BUC11 (SEQ ID NO: 36) has a role in proliferation of cancer cells in the breast.

Isolation and Sequencing of BUC6 (SEQ ID NO: 31) cDNA

After screening half million plaque forming units (pfu) from the breast cDNA library with BUC6 (SEQ ID NO: 31)-specific probe, 6 positive clones were obtained. Inserts sizes of these clones ranged between ˜1.0 and ˜1.9 kb. All the 6 cDNA inserts were sequenced at least partially. This showed that these 6 cDNA inserts have the same 3′ ends with different length poly (A)+ tails. The longest insert (1879 bp) contained an initiation codon, an open reading frame for 87 amino acid residues, a stop codon (TAG), and a 3′ untranslated region. However there was not a stop codon before the initiation codon, suggesting the possibility of additional 5′ coding sequence (FIGS. 7A and 7B). Amino acid sequence homology searches revealed that the first 20 amino acids from the N-terminal region are identical to NY-BR-1 antigen (residues 433-452), whereas the rest of the amino acid residues are not highly homologous to any known proteins (FIG. 7C). Homology studies were carried out using BLASTp.

This is shown in FIG. 30. To summarise, the “BUC gene” mRNA sequence is composed, at its 5′ end, of 2510 bp (including BUC6 (SEQ ID NO: 31) sequence) in the 3′ end of the NY-BR-1 gene but not in the coding region and, at its 3′ end, of the 1308 bp sequence of LOC646360. Basically, LOC646360 matches 50% of BUC11 (SEQ ID NO: 36) sequence.

Using softwares available on the internet, in silico analysis of the LOC646360 predicted protein has been analysed. According to the PSORT II server (http://psort.nibb.ac.jp/form2.html), LOC646360 protein could be a cytoplasmic protein. According to the iPSORT (http://hc.ims.u-tokyo.ac.jp/iPSORT/) prediction, LOC646360 protein has a mitochondrial targeting peptide. Furthermore, the protein pattern according to JustBio PatSearch (http://www.justbio.com/patterns/) is: MYRISTYL pattern (position 55), TYR_PHOSPHO_SITE pattern (24) and PKC_PHOSPHO_SITE pattern (10, 56).

### Discussion

EST data bases are repositories of the human transcriptome, containing a wealth of nucleic acid sequence information and mRNA expression data. EST database mining has resulted in the discovery of a prostate cancer-related gene, PAGE-1/GAGE-B (7), a Ewing's sarcoma-associated gene, XAGE-1 (8), and a number of differentially expressed transcripts in glioblastoma (9). The UniGene resource, developed at NCBI, clusters ESTs and other mRNA sequences, along with coding sequences (CDSs) annotated on genomic DNA, into subsets of related sequences. This study mined UniGene database for gene clusters associated with human breast.

The current bioinformatics analysis identified 228 UniGene clusters associated with human breast. One of them, BUC6 (SEQ ID NO: 31), contains 56 ESTs. Some ESTs are identical to breast cancer antigen NY-BR-1 mRNA but most of them are not homologous to any known genes. Both conventional and real-time RT-PCR analyses confirmed that BUC 6 (SEQ ID NO: 31) is highly expressed in normal breast tissues, testes, and breast cancer tissues and a breast cancer cell line (T47D) but not in 21 different normal tissues. The inventors cloned BUC6 (SEQ ID NO: 31) cDNA from a breast cancer cDNA library.

BUC6 (SEQ ID NO: 31) is also highly expressed in melanoma. BUC9 (SEQ ID NO: 32) is highly expressed in breast cancer tissues and cell lines and is expressed in placenta, testis and breast regarding the normal tissue expression. BUC9 (SEQ ID NO: 32) is also highly expressed in oesophageal (paired normal and cancer) tissues and is expressed in 50% of the gastric (paired normal and cancer) tissues tested. Furthermore, BUC9 (SEQ ID NO: 32) is highly expressed in melanoma. BUC10 (SEQ ID NO: 35) is highly expressed in melanoma and breast cancer cell lines. BUC10 (SEQ ID NO: 35) mRNA expression is lower in breast tumours than in some normal tissues and other cancers. BUC11 (SEQ ID NO: 36) is expressed only in normal breast and normal testis regarding the normal tissue distribution. BUC11 (SEQ ID NO: 36) is highly expressed in breast cancer tissues, testicular cancer tissues but its expression is quite low in the breast cancer cell lines T47D and MDA231. BUC11 (SEQ ID NO: 36) is not expressed in PBMC, mesothelioma tissues, melanoma tissues, gastric tissues, kidney tissues and oesophageal tissues.

Breast cancer is the most common cancer in women and carries a high mortality rate. Detection of axillary lymph node metastases is the most valuable prognostic parameter for breast cancer. The standard technique for detection of the presence of lymph node metastasis is the histological analysis of one or a few hematoxylin and eosin (H&E) sections from each lymph node. However, small foci of metastatic disease are not easy to identify by this technique. As many as 30% of patients considered to have negative nodes ultimately develop recurrent disease (10). PCR combines superb sensitivity with high throughput. Real-time PCR has the ability to quantify gene expression level over a wide range dynamic range. Both conventional and real-time RT-PCR analyses of BUC6 (SEQ ID NO: 31), BUC9 (SEQ ID NO: 32) and BUC11 (SEQ ID NO: 36) could be useful for detection of metastatic breast cancer cells in axillary lymph nodes as well as peripheral blood stream.

Most (about 77%) of women with breast cancer are older than 50 when they are diagnosed (11). Unintentional destruction of normal breast tissues by a given of novel therapeutic strategies for patents older than 50 with advanced breast cancer should have minimal consequences. BUC6 (SEQ ID NO: 31), BUC9 (SEQ ID NO: 32), BUC10 (SEQ ID NO: 35), BUC11 (SEQ ID NO: 36) and their products could be used as a target for gene and/or immune therapy for breast cancer patients.

According to the gene expression analysis, BUC6 (SEQ ID NO: 31), BUC9 (SEQ ID NO: 32) and BUC10 (SEQ ID NO: 35) could be useful in the management of melanoma. Also, BUC9 (SEQ ID NO: 32) and BUC10 (SEQ ID NO: 35) could be useful in the management of oesophageal cancer and gastric cancer. The presence or absence of the splice variants of BUC9 (SEQ ID NO: 32) could be an indication of the disease state for breast cancer. Furthermore, BUC11 (SEQ ID NO: 36) could be useful in the management of testicular cancer and could be of interest for the diagnosis and/or prognosis of breast cancer.

### REFERENCES

1. http://www.cancerresearchuk.org
2. King M C, Marks J H, Mandell J B; New York Breast Cancer Study Group. Breast and ovarian cancer risks due to inherited mutations in BRCA1 and BRCA2. Science. 2003; 302(5645):643-6.
3. Burstein H J. The distinctive nature of HER2-positive breast cancers. N Engl J. Med. 2005; 353:1652-4.
4. http://www.ncbi.nlm.nih.gov/dbEST/index.html
5. Livak K J, Schmittgen T D. Analysis of relative gene expression data using real-time quantitative PCR and the 2(-Delta Delta C(T)) Method. Methods. 2001; 25:402-8.
6. Jager D, Stockert E, Gure A O, Scanlan M J, Karbach J, Jager E, Knuth A, Old L J, Chen Y T. Identification of a tissue-specific putative transcription factor in breast tissue by serological screening of a breast cancer library. Cancer Res. 2001; 61(5):2055-61.
7. Brinkmann U, Vasmatzis G, Lee B, Yerushalmi N, Essand M, Pastan I. PAGE-1, an X chromosome-linked GAGE-like gene that is expressed in normal and neoplastic prostate, testis, and uterus. Proc Natl Acad Sci USA. 1998; 95(18):10757-62.
8. Liu X F, Helman L J, Yeung C, Bera T K, Lee B, Pastan I. XAGE-1, a new gene that is frequently expressed in Ewing's sarcoma. Cancer Res. 2000; 60(17):4752-5.
9. Loging W T, Lal A, Siu I M, Loney T L, Wikstrand C J, Marra M A, Prange C, Bigner D D, Strausberg R L, Riggins G J. Identifying potential tumor markers and antigens by database mining and rapid expression screening. Genome Res. 2000; 10(9):1393-402.
10. Cady B. The need to reexamine axillary lymph node dissection in invasive breast cancer. Cancer. 1994; 73(3):505-8.
11. http://www.cancer.org
12. Sahin U., et al., PNAS (1995), 92(25):11810-3.

1. An isolated mammalian nucleic acid molecule selected from the group consisting of:
a) a nucleic acid sequence encoding BUC11 (SEQ ID NO:36), BUC6 (SEQ ID NO:31), BUC9 (SEQ ID NO:32) and/or BUC10 (SEQ ID NO:35);
b) a nucleic acid sequence comprising a nucleic acid sequence encoding at least one amino acid open reading frame obtainable from a nucleic acid sequence encoding BUC11 (SEQ ID NO:36), BUC6 (SEQ ID NO:31), BUC9 (SEQ ID NO:32) and/or BUC10 (SEQ ID NO:35), wherein a peptide having the amino acid sequence of the encoded reading frame is capable of cross-reacting with samples from patients susceptible to cancer and/or from samples from patients with cancer;
c) a fragment of a nucleic acid molecule as defined in a) or b) comprising at least 20 contiguous nucleotides or which encodes at least 4 amino acids of the sequence;
d) nucleic acid molecules, the complementary strand of which specifically hybridises to nucleic acid molecules described in a), b) or c);
e) nucleic acid molecules, the sequence of which differs from the sequence of a nucleic acid molecule of a), b) c) or d) due to the degeneracy of the genetic code; or
f) nucleic acid molecules comprising at least 10 contiguous nucleotides capable of specifically hybridising to a nucleic acid molecule as defined in a), b), c), d) or e).

2. An isolated nucleic acid molecule which is at least 80% homologous to a nucleic acid sequence as defined in claim 1 and which is expressed in breast tissue but not normal adrenal gland, brain, foetal brain, foetal liver, heart, kidney, liver, lung, placenta, prostate, saliva, skeletal muscle, small intestine, spinal cord, spleen, thymus, thyroid, trachea and/or uterus.

3. An isolated nucleic acid molecule according to claim 2 wherein the expression is mRNA expression.

4. An isolated nucleic acid molecule comprising at least 15 nucleic acids capable of specifically hybridising to a sequence within a nucleic acid molecule according to claim 1.

5. A vector comprising a nucleic acid molecule according to claim 1.

6. A host cell comprising a vector according to claim 5.

7. An isolated polypeptide comprising an amino acid sequence encoded by a nucleic acid molecule according to claim 1.

8. (canceled)

9. A fragment or derivative of a polypeptide according to claim 7 comprising at least 10 contiguous amino acids of the BUC11 (SEQ ID NO:36), BUC6 (SEQ ID NO:31), BUC9 (SEQ ID NO:32) or BUC10 (SEQ ID NO:35) sequences.

10. A monoclonal antibody capable of specifically binding to a polypeptide according to claim 7, or fragment or derivative thereof comprising at least 10 contiguous amino acids of the BUC11 (SEQ ID NO:36), BUC6 (SEQ ID NO:31), BUC9 (SEQ ID NO:32) or BUC10 (SEQ ID NO:35) sequences.

11.-12. (canceled)

13. A method of detecting or monitoring cancer comprising the step of detecting or monitoring of a nucleic acid molecule comprising a sequence according to claim 1 in a sample from a patient.

14. A method of detecting or monitoring cancer comprising the step of using a nucleic acid molecule comprising a sequence according comprising a sequence according to claim 1 or a probe capable of specifically hybridizing the nucleic acid molecule in combination with a reverse transcription polymerase chain reaction (RT-PCR).

15. A method of detecting or monitoring cancer comprising detecting or monitoring elevated levels of a polypeptide according to claim 7.

16. A method according to claim 15 further comprising the step of using an antibody selective for the polypeptide to detect the polypeptide.

17. A method according to claim 16 further comprising the step of using an Enzyme-Linked ImmunoSorbant Assay (ELISA).

18. (canceled)

19. A kit for use with a method according to claim 13 comprising an isolated mammalian nucleic acid molecule selected from the group consisting of:
a) a nucleic acid sequence encoding BUC11 (SEQ ID NO:36), BUC6 (SEQ ID NO:31), BUC9 (SEQ ID NO:32) and/or BUC10 (SEQ ID NO:35);
b) a nucleic acid sequence comprising a nucleic acid sequence encoding at least one amino acid open reading frame obtainable from a nucleic acid sequence encoding BUC11 (SEQ ID NO:36), BUC6 (SEQ ID NO:31), BUC9 (SEQ ID NO:32) and/or BUC10 (SEQ ID NO:35), wherein a peptide having the amino acid sequence of the encoded reading frame is capable of cross-reacting with samples from patients susceptible to cancer and/or from samples from patients with cancer;
c) a fragment of a nucleic acid molecule as defined in a) or b) comprising at least 20 contiguous nucleotides or which encodes at least 4 amino acids of the sequence;
d) nucleic acid molecules, the complementary strand of which specifically hybridises to nucleic acid molecules described in a), b) or c);
e) nucleic acid molecules, the sequence of which differs from the sequence of a nucleic acid molecule of a), b) c) or d) due to the degeneracy of the genetic code; or
f) nucleic acid molecules comprising at least 10 contiguous nucleotides capable of specifically hybridising to a nucleic acid molecule as defined in a), b), c), d) or e); or
a polypeptide comprising an amino acid sequence encoded by the nucleic acid molecule; or
an antibody selective for the polypeptide.

20. (canceled)

21. A method of prophylaxis or treatment of cancer comprising administering to a patient a pharmaceutically effective amount of a nucleic acid molecule hybridisable under high stringency conditions to a nucleic acid molecule comprising a nucleic acid sequence according to claim 1 or a pharmaceutically effective fragment thereof.

22. A method of prophylaxis or treatment of cancer comprising administering to a patient a pharmaceutically effective amount of a polypeptide as defined in claim 7 or a pharmaceutically effective fragment thereof.

23. A method of prophylaxis or treatment of cancer comprising the step of administering to a patient a pharmaceutically effective amount of an antibody according to claim 10.

24. A method according to claim 22, wherein the cancer is a gastro-intestinal cancer or a breast cancer.

25.-26. (canceled)

27. A polypeptide according to claim 7 or a pharmaceutically effective fragment thereof, attached to a carrier protein.
